(19) United States
(12) Patent Application Publication
(10) Pub. No.: US 2016/0151503 A1
(43) Pub. Date: Jun. 2, 2016

(54) COMPOSITIONS AND PREPARATION METHODS OF LOW MELTING IONIC SALTS OF POORLY-WATER SOLUBLE DRUGS

(71) Applicant: MW ENCAP LIMITED, West Lothian (GB)

(72) Inventors: Chris Porter, South Melbourne, Victoria (AU); Peter Scammells, North Balwyn, Victoria (AU); Hywel Williams, Boston, MA (US); Yasemin Sahbaz, Coburg North, Victoria (AU)

(73) Assignee: MW ENCAP LIMITED, West Lothian (GB)

(21) Appl. No.: 14/906,507

(22) PCT Filed: Aug. 1, 2014
(86) PCT No.: PCT/AU2014/050168

§ 371 Date: Jan. 20, 2016

(30) Foreign Application Priority Data
Aug. 1, 2013 (AU) 2013902874

(51) Int. Cl. A61K 47/44; A61K 31/137; A61K 31/496; A61K 9/48; A61K 31/4748; A61K 31/155; A61K 31/192; A61K 31/495; A61K 31/445

(52) U.S. Cl. CPC A61K 47/44; A61K 31/495; A61K 31/137; A61K 31/496; A61K 31/445; A61K 31/4748; A61K 31/155; A61K 31/192; A61K 9/4841

(57) ABSTRACT

The disclosure relates generally to ionic salts, particularly low-melting ionic salts such as ionic liquids, of poorly-water soluble drugs. The disclosure further relates to methods of preparing the ionic salts of poorly-water soluble drugs, lipid formulations comprising them and their use in drug delivery.

### FIELD

The present disclosure relates generally to ionic salts, particularly to low melting salts, such as ionic liquids, of poorly water soluble drugs and their use in drug delivery. The present disclosure relates further to ionic salts, particularly low melting salts, such as ionic liquids, of poorly water soluble drugs and formulations containing them. The disclosure also relates to methods for the preparation of ionic salts, particularly low melting salts, such as ionic liquids, of poorly water soluble drugs, and to methods for the preparation of formulations containing them, as well as dosage forms containing the low melting salts, such as ionic liquids, or formulations thereof.

### BACKGROUND

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

An ionic liquid is an ionic salt in the liquid state. Typically, this refers to ionic salts which have a melting point below about 100° C. Ionic liquids (ILs) have generated considerable recent interest in fields as broad as catalysis, extraction, energy storage and CO₂ capture. The unique solvent properties of ILs are perhaps most well described, and form the basis of the use of ILs, as “green” solvents in chemical synthesis.

A potential drug candidate for oral administration must meet at least three standards to allow effective absorption from the gastrointestinal tract: acceptable stability in the gastrointestinal tract, acceptable membrane permeability and acceptable solubility the gastro-intestinal tract. Once the challenges of acceptable stability and membrane permeability are met, there still remains the need to ensure sufficient quantities of the drug are solubilized in the gastrointestinal fluids to allow flux across the absorptive membrane. In this regard, poorly water-soluble drugs (PWSDs) are a particular challenge in drug delivery.

Drugs that have an acceptable degree of permeability but are poorly water soluble can be categorized as Biopharmaceutical Classification System (BCS) Class II drugs and appropriate choice of formulation will determine whether such a drug will be adequately absorbed. For these molecules, traditional formulations (tablets, capsules etc.) typically fail to provide for useful drug exposure after oral administration. This reflects the fact that in almost all cases, drugs must be molecularly dispersed in aqueous solution in the gastro-intestinal (GI) fluids for absorption to occur. For PWSDs, the process of drug dissolution is usually sufficiently slow that drug absorption is limited. This is an increasingly important problem for the pharmaceutical industry, where the prevalence of PWSDs emerging from drug discovery programs is increasing rapidly, with recent estimates suggesting that up to 90% of prospective development candidates have physicochemical properties that are likely to lead to absorption problems. This is the case for BCS class II drugs (where solubility is the primary limitation), but also extends to BCS class IV drugs where both solubility and permeability limit drug absorption. In both cases, however, a means to enhance effective solubility in the GI tract is a critical determinant of effective exposure after oral administration.

A common mechanism by which the absorption of PWSDs can be enhanced is to pre-dissolve the drug in a non-aqueous liquid vehicle, for example, a lipid, and to ‘piggy-back’ onto endogenous lipid digestion/absorption pathways. This delivers the drug to the intestine in a pre-dissolved, molecularly dispersed form, and molecular dispersion is maintained by continued solubilization in the lipidic microdomains (micelles, vesicles etc) that are produced by the process of lipid digestion. Such formulations are typically referred to as “lipid formulations”, or “lipid-based formulations” and examples thereof include the drug dissolved in simple lipid solutions, self emulsifying drug delivery systems (SEDDS) and even systems that contain very little or no actual lipids, such as co-solvent- and/or surfactant-based formulations.

Notwithstanding the usefulness of this technique, it is nevertheless limited somewhat by the solubility of the drug in the formulation and the desired size of the eventual dosage form. By way of example, a typical lipid based formulation might contain 30-50% by weight lipid. For even the largest capsule the maximum quantity of formulation that can be included is 1000 mg and this, along with the drug solubility in the formulation, places a ‘cap’ on the quantity of drug that can be delivered per capsule. Thus, it may be necessary for a patient to take either multiple dosage forms and/or large dosage forms and/or large dosage forms to ensure administration and absorption of an effective amount of the PWSD, a disadvantage that can lead to poor patient compliance.

### SUMMARY

It has now been found that where a PWSD is converted into a low melting ionic salt, such as an ionic liquid, the PWSD may become substantially more soluble or even miscible in a substantially non-aqueous vehicle, to afford a lipid formulation of the PWSD.

Pre-forming the low melting ionic salt and subsequently blending the pre-formed ionic salt with a substantially non-aqueous vehicle may allow for an increase in solubility and/or miscibility of the PWSD in the vehicle. Advantageously, it may therefore be possible to increase the drug loading into a suitable vehicle when compared to the amount of non-ionised drug that can be dissolved in the same vehicle.

In some embodiments, the formation of a low melting ionic salt may also advantageously increase drug solubility in the colloidal species present in the intestinal tract. This promotes ongoing solubilisation of the ionic salt in the GI fluids as a substantially non-aqueous vehicle is digested and incorporated into endogenous lipid dispersion and solubilisation process. Maintenance of drug in a solubilised state may subsequently promote drug absorption and avoid, reduce or minimize the detrimental effects of drug precipitation. Incorporation into lipid processing pathways also typically reduces the ‘food effect’ commonly seen for poorly water soluble drugs where co-administration with food increases drug absorption but does so in a poorly controlled and clinically variable manner.

In some embodiments, by facilitating the production of a formulation where the drug is dissolved in or miscible with a substantially non-aqueous vehicle, other advantages may also be achieved, such as a reduction in GI irritation, and a reduction in taste (due to a reduction of the concentration of drug in aqueous solution).

Accordingly, in a first aspect, the present disclosure relates to a lipid formulation of a poorly water soluble drug comprising a low melting ionic salt of the poorly water soluble drug, together with a substantially non-aqueous lipid vehicle.

By the use of an appropriate counter ion the low melting ionic salt of the poorly water soluble drug melts at a lower temperature than that of the non-ionised poorly water soluble drug and, dependent upon the nature of the poorly water soluble drug and the counter ion, may melt at a temperature below about 100° C. (also referred to as an ionic liquid salt) or may melt at a temperature of about 100° C. or above.

Thus, one embodiment of the present disclosure relates to a lipid formulation of a poorly water soluble drug comprising an ionic liquid salt of the poorly water soluble drug, together with a substantially non-aqueous lipid vehicle.

In some embodiments, the ionic liquid salt has a melting point of about 90° C. or less. In some further embodiments, the ionic liquid salt has a melting point of about 80° C. or less. In further embodiments, the ionic liquid salt has a melting point of about 70° C. or less. In further embodiments, the ionic liquid salt has a melting point of about 60° C. or less. In further embodiments, the ionic liquid salt has a melting point of about 50° C. or less. In further embodiments, the ionic liquid salt has a melting point of about 40° C. or less. In further embodiments, the ionic liquid salt has a melting point of about 30° C. or less. In still further embodiments, the ionic liquid salt is an oil at room temperature. In yet other embodiments, the ionic liquid salt may have a melting point in the range of about 90-75° C., or about 80-65° C., or about 70-60° C., or about 65-55° C., or about 60-50° C., or about 55-45° C., or about 50-40° C., about 45-35° C., or about 40-30° C.

In some embodiments, the low melting ionic salt is at least 50% more soluble in the non-aqueous lipid vehicle compared to the non-ionised PWSD. In further embodiments, the low melting ionic salt is at least 2-3 times more soluble in the non-aqueous lipid vehicle compared to the non-ionised PWSD. In further embodiments, the low melting ionic salt is at least 4-5 times more soluble in the non-aqueous lipid vehicle compared to the non-ionised PWSD. In still further embodiments, the low melting ionic salt is at least 10 limes more soluble in the non-aqueous lipid vehicle compared to the non-ionised PWSD.

Another embodiment of the present disclosure relates to a lipid formulation of a poorly water soluble drug comprising a low melting ionic salt of the poorly water soluble drug, which salt melts at a temperature of about 100° C. or above, together with a substantially non-aqueous lipid vehicle.

In certain embodiments, the lipid formulation is suitable for oral administration to a patient, for example as a liquid fill for a capsule.

Thus, there is further provided a fixed dosage form, such as a capsule, containing a lipid formulation of a poorly water soluble drug comprising a low melting ionic salt of the poorly water soluble drug, together with a substantially non-aqueous lipid vehicle.

In another aspect, there is provided a method for the manufacture of a lipid formulation of a poorly water soluble drug, said method comprising the step of blending a low melting ionic salt of the poorly water soluble drug with a non-aqueous lipid vehicle.

In some embodiments, the disclosure relates to a method for the manufacture of a lipid formulation of a poorly water soluble drug, said method comprising the step of forming a low melting ionic salt of the poorly water soluble drug and blending the low melting ionic salt of the poorly water soluble drug with a non-aqueous lipid vehicle to form a lipid formulation of the poorly water soluble drug. In a further embodiment, the method comprises the additional step of filling a capsule with the lipid formulation of the poorly water soluble drug.

In some further embodiments of the disclosure there is provided use of a low melting ionic salt of poorly water soluble drug to increase loading of the poorly water soluble drug in a non-aqueous lipid vehicle.

### BRIEF DESCRIPTION OF FIGURES

FIG. 1 graphically compares cinnarizine plasma concentration versus time data after administration of cinnarizine free base (Cin FB) or cinnarizine decylsulfate IL (Cin IL) as either a solution or suspension in a SEDDS formulation (15% w/w soybean oil, 15% w/w Maisine 35-1, 60% w/w Cremophor EL, 10% w/w EtOH) or an aqueous suspension.

FIG. 2 graphically depicts the fate of cinnarizine decylsulfate IL (Cin DS) following dispersion and digestion of the SEDDS solution formulation in simulated intestinal fluid (SIF).

FIG. 3 graphically depicts itraconazole plasma concentration after oral administration of a commercial formulation of itraconazole free base (ITZ FB) or a SEDDS formulation of itraconazole docusate ionic liquid (ITZ IL) at 20 mg/kg itraconazole free base equivalents to rats.

FIG. 4 graphically depicts itraconazole concentration in the aqueous phase of an in vitro digestion experiment that compares solubilisation after digestion of a SEDDS formulation containing itraconazole docusate ionic liquid (ITZ IL) and a comparator formulation containing itraconazole free base (ITZ FB) at the same concentration as a suspension.

### DESCRIPTION

Throughout this specification and the claims which follow, unless the context requires otherwise, the word “comprise” and variations such as “comprises” and “comprising” will be understood to imply the inclusion of a stated integer or step or group of integers but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary, elements of the invention. The phrase allows for the presence of other non-recited elements which do not materially affect the characteristics of the invention but excludes additional unspecified elements which would affect the basic and novel characteristics of the method defined

The singular forms “a”, “an” and “the” include plural aspects unless the context clearly dictates otherwise.

The term “invention” includes all aspects, embodiments and examples as described herein.

As used herein, a “low melting ionic salt” or a “low melting salt” of a poorly water soluble drug refers to an ionic salt of said drug comprised of an ionised form of the drug and corresponding counter ion, wherein the ionic salt has a melting temperature lower than that of the non-ionised drug. In some embodiments, the low melting salts melt at a temperature of about less than 100° C. In other embodiments, the low melting salts melt at a temperature of about 100° C. or above.

It will be understood that reference to a melting point (or melting temperature) is not intended to be limited to a single quantitative value but also includes, as appropriate, ranges of values. In some instances the temperature at which transition from a solid to a molten state may be more accurately referred to as glass transition temperature and it will be understood for the purpose of the present disclosure that this is encompassed by reference to a melting point or melting temperature.

In some embodiments, useful low melting ionic salts are those with a melting point substantially lower than that of the non-ionised drug. Thus, an observed reduction in melting point may be at least about 10° C., 20° C., 30° C., 40° C., 50° C., 60° C., 70° C. 80° C., 90° C. or 100° C. lower than that of the non-ionised drug. Alternatively, the melting point of the low melting ionic salt may be assessed as a % value reduction in the melting point of the non-ionised drug, such as at least about 10%, 20%, 30%, 40%, 50%, 60%, 70%, 80%, 90%, 100% or more. Such a reduction may afford an increase in solubility of the PWSD in a non aqueous vehicle, regardless of the absolute magnitude of the melting point and thus small differences in melting point between the ionised and non-ionised forms, which may include overlapping or narrowed/expanded melting ranges, may nevertheless afford advantages of the disclosure. For non-ionised compounds with high initial melting points, for example at least about 150° C., or at least about 170-180° C., or about 200° C. or greater, a significant relative decrease in melting point may lead to a significant and practically useful increase in solubility in a substantially non aqueous vehicle, even if the absolute melting point of the corresponding ionic salt remains >100° C.

Reference to an ionic liquid salt, or ionic liquid (IL), refers to a low melting ionic salt, typically having a melting point below about 100° C. In some embodiments, the ionic liquid has a melting temperature of about 90° C. or less, or about 80° C. or less, or about 70° C. or less, or about 60° C. or less, or about 50° C. or 40° C. or less, such as about 30° C. or less, such as about 20° C. or less. In certain embodiments, the ionic liquid is a liquid or oil at room temperature (for example, at a temperature of about 18-30° C., such as about 18-25° C.). Thus, an ionic liquid may have a melting point in the range of about 90-75° C., or about 80-65° C., or about 70-60° C., or about 65-55° C., or about 60-50° C., or about 55-45° C., or about 50-40° C., about 45-35° C., about 40-30° C. or about 30-20° C.

Any counter ion which affords a low melting ionic salt of the poorly water soluble drug is encompassed by the present disclosure, Some suitable counter ions are ionised forms of organic (carbon containing) compounds. In some embodiments the ionised forms of organic (carbon containing) compounds are highly lipophilic to promote solubility of the low melting ionic salt formed in lipid vehicles

Where a non-ionised drug is highly insoluble in a lipid vehicle, a improvement in solubility of even several-fold may nevertheless still only result in a small amount of drug being solubilised (e.g. ≦1, ≦5 or ≦10 mg/g on a non-ionised equivalent basis). While such embodiments are contemplated by the disclosure, in other embodiments, the low melting ionic salts of the disclosure advantageously afford a solubility of the PWSD in the non-aqueous lipid vehicle (on a non-ionised equivalent basis) of at least about 20 mg/g, or about 50 mg/g, such as at least about 70-80 mg/g, or at least about 100 mg/g or at least about 150 mg/g or at least about 200-250 mg/g (on a non-ionised drug equivalent basis). In further examples thereof, the low melting ionic salts may demonstrate an increase in solubility of the PWSD in a substantially non-aqueous vehicle compared to that of the non-ionised form. Thus, in some embodiments, the low melting ionic salt may afford an improvement in solubility of the PWSD in the non-aqueous lipid vehicle over the non-ionised drug by at least 20-30%, such as an improvement of at least about 50%, or about 100-200% (2-3 fold improvement). In still further examples, the low melting ionic salts may afford at least about a 4-fold, 5-fold, 6-8-fold or at least about 10-fold improvement in solubility. In still further embodiments, the low melting ionic salts may afford at least about a 20-fold, 30-fold, or at least about 40-50-fold improvement in solubility.

As used herein, “poorly water soluble drug” (PWSD) includes pharmacologically or physiologically active compounds having water solubility of about 100 mg/ml or less. In further examples, the PWSD has a water solubility of about 90 mg/ml, 80 mg/ml, 70 mg/ml, 60 mg/ml, 50 mg/ml, 40 mg/ml, 30 mg/ml, 20 mg/ml, 10 mg/ml, 5 mg/ml, 2 mg/ml or 1 mg/ml, or less. In still further embodiments, the PWSD has a water solubility of about 500 μg/ml or less, such as about 300 μg/ml or less, 100 μg/ml, 50 μg/ml, 25 μg/ml, 10 μg/ml 5 μg/ml or 1 μg/ml or less. It will be understood that the term. “pharmacologically or physiologically active compound” includes any compound which when administered to a subject provides a beneficial effect to said subject, and includes, but is not limited to, disease and disorder preventative and ameliorating agents which interact with the physiology or pharmacology of the subject, agents which interact with infective microorganisms (e.g. viruses and bacteria), and nutritional agents (e.g. vitamins, amino acids and peptides).

In order to form the low melting ionic salt, the PWSD must bear at least one ionisable group or atom capable of forming an ionic pair with a suitable counter ion. The PWSD may form the cation or the anion of the ionic pair.

In some embodiments, the PWSD forms the cation of the ionic pair. In some embodiments thereof, the PWSD contains at least one basic ionisable nitrogen atom that can form a quaternary nitrogen atom. In some embodiments, quaternary nitrogen atoms may be prepared by protonation or alkylation of the nitrogen atom. Suitable methods therefor are known in the art. Said nitrogen atom may be present in the molecule as a primary amine group (—NH₂) or secondary or tertiary amine (mono or disubstituted amino) group, or part of a saturated or unsaturated ring moiety (for example, part of a pyrrolidine, pyrrole, pyrroline, pyrazole, imidazole, triazole, tetrazole, oxazole, thiazole, pyrazoline, imidazoline, pyrazolidine, imidazolidine, piperidine, piperazole, pyridine, pyrimidine, pyrazine, pyridazine, morpholine, thiomorpholine, azepine, indole, isoindole, indoline, isoindoline, indazole or benzimidazole moeity) within the PWSD. In some embodiments, the ionisable nitrogen atom is part of an amino acid or amino acid residue, such as within a peptide.

Where the PWSD bears an ionisable group or atom, such as a nitrogen atom, which is ionised to form a positively charged cation, the counter anion is a negatively charged ion (anion).

In certain embodiments, the counter ion is selected from anions formed from carboxylic acids (RC(O)O⁻), phosphates (ROP(O)O₂⁻), phosphonates (RP(O)O₂⁻), sulfonates (RSO(O)₂O⁻), sulfates (ROS(O)₂O⁻), tetrazolyls (R-tetrazolate) and bis(sulfonyl)imides (RSO₂—N⁻—SO₂R) where R may be any suitable group, such as an optionally substituted hydrocarbon group. In some further embodiments, the hydrocarbon group may have at least 2 carbon atoms. In some further embodiments, the counter ion is a sulfate (SO₄R). In some further embodiments of suitable anions, R has at least 4 carbon atoms. In still further embodiments, R has from 6-10 or 11-18 or 19-24 carbon atoms.

In some embodiments, R is alkyl. As used herein, “alkyl” may be a saturated straight chained or branched hydrocarbon. In some embodiments, “alkyl” refers to a hydrocarbon group having from 4-40 carbon atoms, such as from 4-24 carbon atoms, including ranges of from 8-12, 13-16, 17-20, 20-24 and 25-30 carbon atoms. In some embodiments, “alkyl” refers to C1, C2, C3, C4, C5, C6, C7, C8, C9, C10, C11, C12, C13, C14, C15, C16, C17, C18, C19, C20 C21, C22, C23 or C24 straight or branched hydrocarbons. In still further embodiments, R has at least 8, 9, 10, 11, 12, 13, 14, 15, 16, 17, 18, 19, 20, 21, 22, 23 or 24 carbon atoms.

In some embodiments, R is a saturated cyclic hydrocarbon (cycloalkyl). The cycloalkyl group may be monocyclic, or polycyclic, including bicyclic or tricyclic fused or bridged ring systems (e.g. norpinane, norbornane and adamantane). In some embodiments thereof, R is a C3, C4, C5, C6, C7, C7, C8, C9 or C10 cycloalkyl group, such as cyclopropyl, cyclobutyl, cyclopentyl or cyclohexyl.

In other embodiments, R is alkenyl or alkynyl, wherein R is a straight chained or branched hydrocarbon group having at least one (for example, 1, 2, 3, 4, 5, 6 or more) double or triple bonds respectively, or a combination of both. In some embodiments, “alkenyl” or “alkynyl” refers to an unsaturated hydrocarbon group having from 4-40 carbon atoms, such as from 4-24 carbon atoms, including ranges of from 8-12, 13-16, 17-20, 20-24 and 25-30 carbon atoms. In further embodiments, alkenyl or alkynyl refers to C2, C3, C4, C5, C6, C7, C8, C9, C10, C11, C12, C13, C14, C15, C16, C17, C18, C19, C20, C21, C22, C23 or C24 hydrocarbons.

In other embodiments, R is an unsaturated cyclic hydrocarbon group having at least one (for example, 1, 2, 3, 4, 5, 6 or more) double (cycloalkenyl) or triple bonds (cycloalkynyl) or a combination of both as permitted by steric constraints. The cycloalkyl group may be monocyclic, or polycyclic, including bicyclic or tricyclic fused or bridged ring systems In some embodiments thereof, R is a C3, C4, C5, C6, C7, C7, C8, C9, C10 cycloalkyl group.

The unsaturated cyclic hydrocarbon group may be aromatic or non-aromatic. In some embodiments, R may include monocyclic or polycyclic aromatic groups such as phenyl or naphthyl.

The R group as described herein may be unsubstituted or may be substituted by 1, 2, 3, 4, 5, or 6 or more same or different optional substituents. Any substituent(s) which have the effect of overall lowering the melting point of the ionic liquid, and/or increasing the solubility of the ionic liquid, typically by increasing lipophilicity (as determined, for example, by comparative log P values), are contemplated. Examples of optional substituents may be selected from C₁₋₆alkyl, C₃₋₆cycloalkyl, phenyl, C₁₋₆alkylphenyl, halo (chloro, fluoro, bromo, iodo), and C(O)alkyl. In some further embodiments, R may be substituted by 1, 2, 3, 4, or more fluoro substituents.

In some further embodiments R is a diester group, derived from a saturated dicarboxylic acid, for example, R′—O(C═O)—(CH₂)ₙ—C(═O)—OR′, where n is from 1-24, such as, 1 (malonate), 2 (succinate), 3 (glutarate), 4 (adipate), 5, 6, 7, 8, 9, 10, 11 or 12. 16 or 20 and R′ is alkyl, cycloalkyl, alkenyl or alkynyl as described above, and may be attached to the carboxylic, phosphate, sulfonate, or sulphate group through, one of the carbon atoms linking the carboxylic groups. In other embodiments, R is a diester group derived from an unsaturated dicarboxylic acid, for example where one, two or three or more pairs of adjacent CH₂ groups are replaced by a —C═C— group. Some examples thereof include cis and trans isomers of R′—O(C═O)—(CH)ₙ—C═C—(CH₂)ₘ—C(═O)—OR′, where n and m are independently selected from 0, 1, 2, 3, 4, 5, 6, 7, 8, 9, 10, 11, 12, 13, 14, 15, 16, 17, 18, 19, 20, 21, and 22, such that m+n=0, 1, 2, 3, 4, 5, 6, 7, 8, 9, 10, 11, 12, 13, 14, 15, 16, 17, 18, 19, 20, 21, or 22. Examples thereof include maleate and fumarate.

In other embodiments, the PWSD forms the anion of the ionic pair and bears an ionisable group or atom such as acidic group, such as a carboxylic, sulphonic or phosphonic acid, sulfate or phosphate group, capable of forming an ionic salt with a positive ion. Such anions can be formed using methods known in the art, for example deprotonation by an appropriate base.

Where the PWSD bears an ionisable group or atom, which is ionised to form a negatively charged anion, the counter anion is a positively charged ion (cation).

In some embodiments the positive ion is a tetraammonium ion, such as ⁺NR′₄, where each R′ is independently selected from hydrogen and hydrocarbon groups, R″, where R″ is as for R defined above, or two R″ groups together with the nitrogen atom form a saturated or unsaturated, including aromatic and non-aromatic, N-containing cyclic group, for example a 5-6 membered monocyclic group, or a fused 9-10-membered bicyclic group. Some examples include ⁺NH₄, ⁺NH₃R″, ⁺NH₂R″, ⁺NHR″₃, wherein each R″ is independently C₄-C₄₀alkyl, C₄-C₄₀alkenyl, or C₄-C₄₀alkynyl, as described above, and which may be optionally substituted as defined for R above. Other examples include cyclic, saturated or unsaturated, aromatic or non-aromatic groups, for example, benzC₁₋₆alkylammonium (e.g benzalkonium), alkylpyridinium ions and dialkylimidazolium ions such as 1-butyl-3-methylimidazolium or 1-hexyl-3-methylimidazolium.

In other embodiments, the positive ion is a phosphonium ion, such as ⁺PR′₄, wherein each R′ is independently selected from hydrogen and hydrocarbon groups (R″) as defined above, or two R″ groups together with the phosphorous atom form a cyclic group. Some examples include ⁺PH₄, ⁺PH₃R″, ⁺PH₂R″₂, ⁺PR″₄, wherein each R″ is independently C₄-C₄₀ alkyl, C₄-C₄₀alkenyl, or C₄-C₄₀alkynyl which may be optionally substituted as defined herein.

Some exemplary, but not limiting, anionic and cationic counterions contemplated by the disclosure are set out in Example 1 and Tables 1-10, and include decylsulfate, lauryl(dodecyl)sulfate, octadecylsulfate, 7-Ethyl-2-methyl-4-undecylsulfate, oleate, triflimide, laurylsulfate, dioctylsulfosuccinate (docusate), dodecylsulfate, saccharinate, methylcyclohexylsulfate, adamantylsulfate, 3,7-dimethyloctanesulfate, octylsulfonate, nonylsulfate, 2-methylcyclohexylsulfate, 5-undecyltetrazolate, butylammonium, octylammonium, dodecylammonium, 1-octyl-3-methylpyridium, 1-hexadecyl-3-methylpyridinium, dimethyl-butyl dodecylammonium, dimethyl-decyl dodecylammonium, decylpyridinium, hexadecyl-trimethylammonium and benzalkonium.

It will understood that some PWSDs may have more than one ionisable group or atom (which may be the same or different) and that one, some or all may be ionised in the formation of the low melting ionic salt. For example a PWSD may have two or three (same or different) ionisable nitrogen atoms or two or three (same or different) ionisable acidic groups. Where more than one atom or group is ionised, each may have the same counter ion or a different counter ion.

Mixtures of ionic salts are also contemplated, for example where, two or three different counter ions are used to form low melting ionic salts of the PWSD with a single ionisable group or atom, where the mixture of ionic salts may be prepared by reacting the ionised PWSD with two or three counter ions. Mixtures of ionic salts may also be prepared by blending or mixing ionic salts.

Any PWSD which can form a low melting ionic salt with a suitable counter ion is contemplated herein. Examples of PWSDs encompassed by the disclosure include those which can be classified within Biopharmaceutical Classification System (BCS) classes II (high in vivo permeability, low aqueous solubility) and IV (low in vivo permeability, low aqueous solubility). Thus in some embodiments, PWSDs contemplated by the disclosure include those classified within Biopharmaceutical Classification System (BCS) class II. In other embodiments, PWSDs contemplated by the disclosure include those classified within Biopharmaceutical Classification System (BCS) class IV. In some embodiments, certain PWSDs, for example, 7-[2-[4-[4-(methoxyethoxy)phenyl]-piperazinyl]ethyl]-2-(furanyl)-7Hpyrazolo[4,3-e]triazolo[1,5-c]pyrimidin-5-amine, are excluded.

The PWSDs are formulated in a substantially non-aqueous lipid vehicle (also referred to herein as a “lipid vehicle”) to provide a lipid formulation. As referred to herein, the substantially non-aqueous lipid vehicle refers to a substantially non-aqueous vehicle which typically contains one or more lipid components, although vehicles containing surfactant, with or without co-solvent, but no lipid/oil component, as described below, may also be considered to be lipid vehicles for the purpose of the disclosure. Thus, reference to a “lipid formulation” is also to be understood that the formulation containing the low melting ionic salt of the PWSD may or may not actually contain a lipid/oil component. The lipid vehicles and resulting lipid formulations may be usefully classified as described below according to their shared common features according to the lipid formulation classification system (LFCS) (Pouton, C. W., Eur. J. Pharm. Sci. 11 (Supp 2), S93-S98, 2000; Pouton, C. W., Eur. J. Pharm. Sci. 29 278-287, 2006).

Thus lipid vehicles, and the resulting lipid formulations, may contain oil/lipids and/or surfactants, optionally with co-solvents. Type I formulations include oils or lipids which require digestion, such as mono, di and tri-glycerides and combinations thereof. Type II formulations are water-insoluble SEDDS which contain lipids and oils used in Type I formulations, with additional water insoluble surfactants. Type III formulations are SEDDS or self-microemulsifying drug delivery systems (SMEDDS) which contain lipids and oils used in Type I formulations, with additional water-soluble surfactants and/or co-solvents (Type IIIa) or a greater proportion of water-soluble components (Type IIIb). Type IV formulations contain predominantly hydrophilic surfactants and co-solvents (e.g. PEG, propylene glycol and diethylene glycol monoethyl ether) and are useful for drugs which are poorly water soluble but not lipophilic. Any such lipid formulation (Type I-IV) is contemplated herein.

Thus, in some embodiments, the lipid formulation comprises a low melting ionic salt, such as an ionic liquid salt, of the poorly water soluble drug, together with one or more oils and/or lipids and optionally one or more surfactants and/or (co)solvents. In some embodiments, the lipid formulation consists essentially of a low melting ionic salt, such as an ionic liquid salt, of the poorly water soluble drug, together with one or more oils and/or lipids and optionally one or more surfactants and/or (co)solvents. In further examples thereof, the lipid formulation comprises a low melting ionic salt, such as an ionic liquid salt, of the poorly water soluble drug, together with one or more oils and/or lipids. In further examples thereof, the lipid formulation consists essentially of a low melting ionic salt, such as an ionic liquid salt, of the poorly water soluble drug, together with one or more oils and/or lipids.

In some embodiments, the lipid vehicle contains one or more oils or lipids, without additional surfactants, co-surfactants or co-emulsifiers, or co-solvents, that is to say consists essentially of one or more oils or lipids. In some further embodiments the lipid vehicle contains one or more oils or lipids together with one or more water-insoluble surfactants, optionally together with one or more co-solvents. In some further embodiments, the lipid vehicle contains one or more oils or lipids together with one or more water-soluble surfactants, optionally together with one or more co-solvents. In some embodiments, the lipid vehicle contains a mixture of oil/lipid, surfactant and co-solvent. In some embodiments, the lipid vehicle is consists essentially of one or more surfactants/co-surfactants/co-emulsifiers, and/or solvents/co-solvents. In some embodiments, resulting the lipid formulation is an oil/lipid-containing formulation, for example any one of Types I, II or III.

In some of the aspects and embodiments described herein, the lipid vehicle consists essentially of water immiscible components, i.e. doesn't not contain any aqueous liquid or water miscible component.

Examples of oils or lipids which may be used in the present invention include almond oil, babassu oil, blackcurrant seed oil, borage oil, canola oil, castor oil, coconut oil, cod liver oil, corn oil, cottonseed oil, evening primrose oil, fish oil, grape seed oil, mustard seed oil, olive oil, palm kernel oil, palm oil, peanut oil, rapeseed oil, safflower oil, sesame oil, shark liver oil, soybean oil, sunflower oil, walnut oil, wheat germ oil, avocado oil, bran oil, hydrogenated castor oil, hydrogenated coconut oil, hydrogenated cottonseed oil, hydrogenated palm oil, hydrogenated soybean oil, partially hydrogenated soybean oil, hydrogenated vegetable oil, caprylic/capric glycerides, fractionated triglycerides, glyceryl tricaprate, glyceryl tricaproate, glyceryl tricaprylate, glyceryl tricaprylale/caprate, glyceryl tricaprylate/caprate, glyceryl tricaprylate/caprate/laurate, glyceryl tricaprylate/caprate/linoleate, glyceryl tricaprylate/caprate/stearate, glyceryl trilaurate, glyceryl monolaurate, glyceryl behenate, glyceryl monolinoleate, glyceryl trilinolenate, glyceryl trioleate, glyceryl triundecanoate, glyceryl tristearate linoleic glycerides, saturated polyglycolized glycerides, synthetic medium chain triglycerides containing primarily C₈-C₁₂ fatty acid chains, medium chain triglycerides containing primarily C₈-C₁₂ fatty acid chains, long chain triglycerides containing primarily >C₁₂ fatty acid chains, modified triglycerides, fractionated triglycerides, and mixtures thereof.

Examples of mono and diglycerides which may be used in the present invention include glycerol mono- and diesters having fatty acid chains from 8 to 40 carbon atoms, including hydrolysed coconut oils (e.g. Capmul® MCM), hydrolysed corn oil (e.g. Maisine™ 35-1). In some embodiments, the monoglycerides and diglycerides are mono- or di-saturated fatty acid esters of glycerol having fatty acid chains of 8 to 18 carbon chain length (e.g. glyceryl monostearate, glyceryl distearate, glyceryl monocaprylate, glyceryl dicaprylate, glyceryl monocaprate and glyceryl dicaprate).

Suitable surfactants for use in the lipid formulations include propylene glycol mono- and di-esters of C₈-C₂₂ fatty acids, such as, but not limited to, propylene glycol monocaprylate, propylene glycol dicaprylate, propylene glycol monolaurate, sold under trade names such as Capryol® 90, Labrafac® PG, Lauroglycol® FCC, sugar fatty acid esters, such as, but not limited to, sucrose palmnitate, sucrose laurate, surcrose stearate; sorbitan fatty acid esters such as, but not limited to, sorbitan laurate, sorbitan palmitate, sorbitan oleate; polyoxyethylene sorbitan fatty acid esters such as, but not limited to, polysorbate 20, polysorbate 40, polysorbate 60, and polysorbate 80, polysorbate 85; polyoxyethylene mono- and di-fatty acid esters including, but not limited to polyoxyl 40 stearate and polyoxyl40 oleate; a mixture of polyoxyethylene mono- and di-esters of C₈-C₂₂ fatty acids and glyceryl mono-, di-, and tri-esters of C₅-C₂₂ fatty acids as sold under tradenames such as Labrasol®, Geluciere® 44/14, Gelucire® 50/13, Labrafil®; polyoxyethylene castor oils compound such as, but not limited to, polyoxyl 35 castor oil, polyoxyl 40 hydrogenated castor oil, and polyoxyl 60 hydrogenated castor oil, as are sold under tradenames such as Cremophor®/Kolliphor EL, Cremophor®/Kolliphor® RH40, Cremophor®/Kollipohor® RH60; polyoxyethylene alkyl ether including but not limited to polyoxyl 20 cetostearyl ether, and polyoxyl 10 oleyl ether; DL-.alpha.-tocopheryl polyethylene glycol succinate as may be sold under the tradename; glyceryl mono-, di-, and tri-ester; a glyceryl mono-, di-, and tri-esters of C₈-C₂₂ fatty acid; a sucrose mono-, di-, and tri-ester; sodium dioctylsulfosuccinate; polyoxyethylene-polyoxypropyene copolymers such as, but not limited to poloxamer 124, poloxamer 188, poloxamer 407; polyoxyethyleneethers of C₈-C₃₂ fatty alcohols including, but not limited to polyoxyethylenelauryl alcohol, polyoxyethylenecetyl alcohol, polyoxyethylenestearyl alcohol, polyoxyethyleneoleyl alcoholas sold under tradenames such as Brij® 35, Brij® 58, Brij® 78Brij® 98, or a mixture of any two or more thereof.

A co-emulsifier, or co-surfactant, may be used in the formulation. A suitable co-emulsifier or co-surfactant may be a phosphoglyceride; a phospholipid, for example lecithin; or a free fatty acid that is liquid at room temperature, for example iso-stearic acid, oleic acid, linoelic acid, linolenic acid, palmitic acid, stearic acid, lauric acid, capric acid, caprylic acid and caproic acid.

Suitable solvents/co-solvents include ethanol, propylene glycol, polyethylene glycol, diethylene glycol monoethyl ether and glycerol.

A polymer may also be used in the formulation to inhibit drug precipitation. A range of polymers have been shown to impart these properties and are well known to those skilled in the art. Suitable polymers include hydroxypropylmethylcellulose, hydroxypropylmethylcellulose acetyl succinate, other cellulose-derived polymers such as methylcellulose; poly(meth)acrylates, such as the Eudragit series of polymers, including Eudragit E100, polyvinylpyrrolidone or others as described in e.g. Warren et al Mol. Pharmaceutics 2013, 10, 2823-2848.

Formulations may also contain materials commonly known to those skilled in the art to be included in lipid based formulations, including antioxidants, for example butylated hydroxyanisole (BHA) or butylated hydroxytoluene (BHT) and solidifying agents such as microporous silica, for example magnesium alumino-metasilicate (Neusilin).

In some embodiments, the lipid vehicle is a SEDDS formulation, typically comprising one or more lipids/oils, one or more surfactants and optionally one or more co-solvents. In further embodiments thereof, the lipid vehicle comprises an oil/lipid phase, a surfactant and ethanol. In further examples thereof the lipid vehicle comprises one or more oils/lipids (such as soy bean oil, and hydrolysed corn oil (C18 monoglyceride and/or diglyceride mixtures, such as glycerol monolinoleate e.g. Maisine™ 35-1)), polyethoylated castor oil (e.g. Cremaphor) and ethanol. In other examples, the lipid vehicle comprises hydrolysed coconut oil (e.g. Capmul), glyceryl tricaprylate/tricaprate (e.g. Captex), polyethoylated castor oil (e.g. Cremaphor) and ethanol. Further suitable examples of SEDDS formulations are described in the Examples herein and may be applied to any low melting ionic salt according to the disclosure.

While formulations containing about ≦% (w/w) or ≦2% (w/w) low melting ionic salt are within the scope of the disclosure, in some embodiments, the lipid formulations contain at least about 5 or about 10 (w/w) % low melting ionic salt, that is to say, at least about 50 or about 100 mg low melting ionic salt per gram of lipid vehicle. In further embodiments, the lipid formulations contain at least about 15 (w/w) %, such as about at least 20 (w/w) % low melting ionic salt, or 25 (w/w) % low melting ionic salt, or 30 (w/w) % low melting ionic salt, or 35 (w/w) % low melting ionic salt or 40 (w/w) % low melting ionic salt, or 45 (w/w) % low melting ionic salt, or 50 (w/w) % low melting ionic salt or 60 (w/w) % low melting ionic salt. In further embodiments, the lipid formulation contains at least about 70 (w/w) % low melting ionic salt or at least about 80 (w/w) % low melting ionic salt.

The lipid formulations and vehicles are substantially non-aqueous, by which is meant that the lipid formulation or lipid vehicle contains less than about 5% water, such as less than 3% or 2%. In further embodiments, the lipid formulation or lipid vehicle contains less than 1% or 0.5%, or does not contain a detectable amount of water.

The lipid formulations may be conveniently prepared by mixing or blending the components of the lipid vehicle, together with the low melting ionic salt of the poorly water soluble drug. Pre-forming the low melting ionic salt prior to mixing or blending with the lipid vehicle affords approximately stoichiometric quantities of the ions and thus may improve solubility. Thus, lipid formulations of the disclosure may advantageously comprise approximately 1:1 stoichiometric quantities of counter ion for each ionised group or atom of the PWSD. Methods for the preparation of low melting ionic salts are known in the art and some exemplary methods, which may be extrapolated to other drugs/counter ions, are described in the Examples. While it may be possible to form the ionic salt in situ, pre-forming the ionic salt also avoids the presence of basifying or acidifying agents in the lipid formulation. Furthermore, for some combinations of PWSD and counter ions efficient in situ formation of low melting ionic salts is not possible. In further embodiments, where the lipid vehicle comprises more than one component, said components may be first blended together before blending with the low melting ionic salt, or alternatively, one or more components of the vehicle may be pre-blended with the low melting ionic salt and the resulting mixture then blended with the remaining components to form the lipid formulation. In certain embodiments, the resulting lipid formulation is a homogenous, single-phase.

Thus, another aspect of the disclosure provides a method for preparing a lipid formulation of a poorly water soluble drug comprising the step of blending a low melting ionic salt of the poorly water soluble drug with a non-aqueous lipid vehicle.

The resulting lipid formulations of the disclosure may be liquid, semi-solid or solid at room temperature. Where the melting point of the ionic liquid salt and/or the lipid vehicle is such that one or more components is solid or semi-solid at room temperature, it may in some embodiments be advantageous to first melt the solid or semi-solid component(s) prior to mixing, and/or maintain an elevated temperature (greater than room temperature—for example, around or above the melting point of the highest melting component) during the mixing process such that the components and formulation remain liquid. In further embodiments, an elevated temperature of the formulation is maintained such that the lipid formulation remains liquid during the process of filling capsules, ampoules, sachets, bottles etc.

The ability to improve the solubility or miscibility of a drug (as the low melting ionic salt, compared to the non ionised form) into a liquid formulation advantageously may allow for increased dosage amount and/or reduced dosage form size and/or number of dosage administrations. In some embodiments, by converting a PWSD into a suitable ionic liquid salt, the amount of drug which may be incorporated or loaded into a lipid vehicle may be at least 2×, or 3×, or 4× or 5×, or 10×, or 25× or 50× 100× or 200× that which may be achieved for the non-ionised form of the drug in the same vehicle. In addition, increasing the dosage amount of the drug may not only allow for improved absorption when administered orally to a patient, but advantageously, may also allow for reduced amounts of surfactant and/or co-solvent to be used in the formulation compared to other formulations used to dissolve the non-ionised PWSD.

Thus, while in some embodiments the lipid formulation or lipid vehicle may consist essentially of surfactant and/or co-surfactant or co-emulsifier, and/or solvent/co-solvent, in other embodiments, the lipid formulation or lipid vehicle contains less than or equal to 50 wt % surfactants, such as less than or equal to 40, or 30, or 25 or 20 or 10, 5, 2% or 1% wt % surfactants. In further embodiments, the lipid formulation or lipid vehicle contains no surfactant. In some embodiments, the lipid formulation or lipid vehicle contains less than or equal to 10 wt % co-solvent, such as less than or equal to 7 or 5 or 2 or 1% co-solvent. In still further embodiments the lipid formulation or lipid vehicle contains no co-solvent.

In some embodiments, the lipid formulation consists essentially of a low melting ionic salt, such as an ionic liquid salt, of the poorly water soluble drug, together with one or more surfactants and/or solvents, optionally with one or more, co-surfactants or co-emulsifiers.

The formulations may be presented in any form suitable for oral administration to a subject. In some embodiments, the lipid formulation is presented in a hard or soft capsule shell. Soft shell capsules or sealable hard shell capsules may be particularly useful for the lipid-based formulations described herein. The capsule shell may be made from any suitable material known therefor. Suitable materials for the capsule shell include gelatin, polysaccharides, and modified starches, and modified celluloses such as hydroxypropylmethylcellulose (HPMC). In other embodiments, the lipid formulation may be presented in container such as a sachet, ampoule, syringe or dropper device or tube or bottle, (for example, a tube or bottle which can be squeezed to deliver its contents), optionally as a fixed dosage, the contents of which may be taken directly or mixed or dispersed into food or liquid. In other embodiments, the lipid formulation may adsorbed onto a suitable solid carrier, such as lactose or silica, which may be filled into a capsule shell or taken directly or mixed in with, or sprinkled onto food or liquid as above.

Subjects contemplated herein include human subjects as well as animal subjects (including, primates; livestock animals such as cows, horses, pigs, sheep and goats; companion animals such as cats, dogs, rabbits, guinea pigs), and, accordingly, in some embodiments, the formulations may be suitable for veterinary purposes.

Some embodiments of the disclosure will now be described with reference to the following examples which are provided for the purpose of illustration only and are not to be construed as limiting the generality hereinbefore described.

### EXAMPLES

### Example 1

### Preparation and Characterisation of Low Melting Ionic Salts

Low melting ionic salts may be prepared according to, or by methods analogous to, the exemplary applications described below as Methods #1-5 by using the appropriate drug and counter ion.

### Methods for Basic Drugs

### Method #1

Developed for counter ions which are slightly soluble in organic solvents.
Drugs: Applicable to drugs which are soluble in organic solvents (e.g. chloroform) such as cinnarizine.HCl and halofantrine.HCl.

### Example Application: Cinnarizine Decylsulfate

Cinnarizine (5.83 g, 15.83 mmol) was dissolved in diethyl ether (300 mL) and a solution of HCl (2M in diethyl ether, 7.92 ml, 15.83 mmol) was added dropwise via a syringe. An off-white precipitate was formed immediately. The resulting precipitate was collected via suction filtration, washed with diethyl ether and dried under vacuum. The resultant cinnarizine.HCl salt (6.35 g, 15.68 mmol) was dissolved in CHCl₃ (500 mL) and decylsulfate ammonium salt (4.01 g, 15.68 mmol) was added. The obtained suspension was refluxed for 2 days. The reaction mixture was cooled to room temperature and washed with distilled water (4×300 mL) until a negative AgNO₃ test was obtained. The organic phase was then dried (anhydrous MgSO₄), filtered and evaporated to afford the desired product (oil) which was dried at 60° C. under high vacuum. Yield 96%.

### Method #2

Developed for water-soluble counter ions (or slightly organic soluble)
Advantage: Shorter reaction time compared with Method #1, Reaction proceeds at room temperature
Used for particularly water-soluble counter ions such as decylsulfate ammonium, dodecylsulfate sodium, octadecylsulfate ammonium salts
Drugs: Applicable to drugs which are soluble in organic solvents (e.g. dichloromethane, chloroform) such as cinnarizine.HCl and halofantrine.HCl.

### Example Application: Cinnarizine Octadecylsulfate

Cinnarizine.HCl salt (2.24 g, 5.54 mmol) was dissolved in DCM (100 mL) and octadecylsulfate ammonium salt (2.04 g, 5.54 mmol) was dissolved in distilled water (100 ml). The two solutions were mixed and the obtained biphasic solution was stirred vigorously for 3 hours. The DCM phase was separated and the aqueous phase was extracted with DCM (2×50 mL). The collected DCM phases were washed with distilled water (3×100 mL) until a negative AgNO₃ test was obtained. The organic phase was then dried (anhydrous MgSO₄), filtered and evaporated to afford the desired product that was dried at 60° C. under high vacuum. Yield 92%.

### Method #3

Developed for water-insoluble counter ions. Also useful for compounds with high water sensitivity
Particularly useful method for counter ions that are insoluble in water but soluble in methanol such as sodium oleate and dioctylsulfosuccinate
Drugs: Applicable to drugs such as cinnarizine.HCl, halofantrine.HCl and itraconazole.HCl

### Example Application: Cinnarizine Oleate

Cinnarizine.HCl salt (87.7 mg, 0.22 mmol) and sodium oleate (65.9 mg, 0.22 mmol) were dissolved in methanol (10 ml) and the clear solution was stirred for 3 hours. Methanol was removed using a rotary evaporator followed by addition of DCM or chloroform (10 mL) to the slurry formed on evaporation. A white precipitate was formed immediately. The resulting precipitate (NaCl) was filtered and organic phase was washed with distilled water (unless the product is water soluble and sensitive) until a negative AgNO₃ test was obtained. The organic phase was then dried (anhydrous MgSO₄), filtered and evaporated to afford the desired product which was dried at 60° C. under high vacuum. Yield 94%.

### Examples of Low Melting Ionic Salts of Basic Drugs

### Cinnarizine Decylsulfate

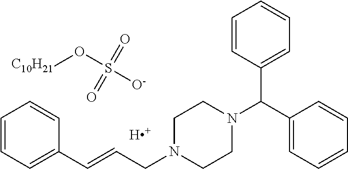

Method #1 and Method #2 have been used to make cinnarizine decylsulfate.

¹H NMR (DMSO-d₆, 400 MHz) δ 9.45 (br s, 1H), 7.51-7.21, (m, 15H), 6.83 (d, J=15.6 Hz, 1H), 6.32 (dt, J=15.6, 7.2 Hz, 1H), 4.48 (s, 1H), 3.91 (d, J=7.2 Hz, 2H), 3.69 (t, 1=6.6 Hz, 2H), 3.33 (br s, 2H), 3.18 (br s, 2H), 2.88 (br s, 2H), 2.26 (br s, 2H), 1.48 (quin, J=6.6 Hz, 2H), 1.28-1.23 (br s, 14H), 0.85 (t, J=6.7 Hz, 3H), HRMS +ve calcd 369.2325, found 369.2314; −ve calcd 237.1155, found 237.1167.

### Cinnarizine Dodecylsulfate

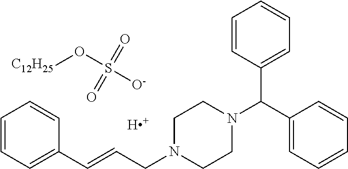

Method #2 and Method #3 have been used to make cinnarizine dodecylsulfate.

¹H NMR (CDCl₃, 400 MHz) δ 10.07 (br s, 1H), 7.44-7.16 (m, 15H), 6.80 (d, J=15.8 Hz, 1H), 6.39 (dt, J=16.0, 7.2 Hz, 1H), 4.36 (s, 1H), 4.10 (t, J=6.8 Hz, 2H), 3.88 (d, J=7.2 Hz, 2H), 3.48 (br s, 2H), 2.96 (br s, 4H), 2.62 (br s, 2H), 1.69 (quin, J=6.8 Hz, 2H), 1.37-1.24 (br s, 18H), 0.89 (t, J=6.8 Hz, 3H), HRMS +ve calcd 369.2331, found 369.2333; −ve calcd 265.1474, found 265.1482.

### Cinnarizine Octadecylsulfate

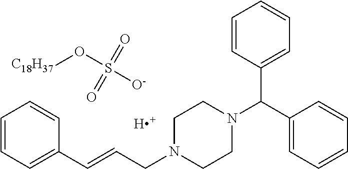

Method #2 has been used to make cinnarizine octadecylsulfate.

¹H NMR (DMSO-d₆, 400 MHz) δ9.48 (br s, 1H), 7.51-7.19 (m, 15H), 6.80 (d, J=15.9 Hz, 1H), 6.31 (dt, J=15.6, 7.2 Hz, 1H), 4.46 (s, 1H), 3.88 (br s, 2H), 3.66 (t, J=6.7 Hz, 2H), 3.33 (br s, 2H), 3.12 (br s, 2H), 2.87 (br s, 2H), 2.24 (br s, 2H), 1.47 (quin, J=6.8 Hz, 2H), 1.29-1.18 (br s, 30H), 0.85 (t, J=6.8 Hz, 3H), HRMS +ve calcd 369.2331, found 369.2333; −ve calcd 349.2413, found 349.2422.

### Cinnarizine 7-ethyl-2-methyl-4-undecyl sulfate

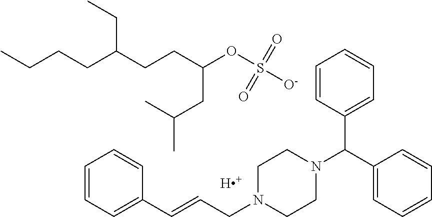

Method #2 and Method #3 have been used to make cinnarizine 7-ethyl-2-methyl-4-undecyl sulfate.

¹H NMR (CDCl₃, 400 MHz) δ10.54 (br s, 1H), 7.43-7.16 (m, 15H), 6.76 (d, J=15.8 Hz, 1H), 6.40 (dt, J=16.0, 7.2 Hz, 1H), 4.51 (m, J=7.8, 5.4 Hz, 1H) 4.36 (s, 1H), 3.85 (d, J=7.4 Hz, 2H), 3.50 (d, J=10.9 Hz, 2H), 2.94 (d, J=10.1 Hz, 4H), 2.67 (br s, 2H), 1.86 (m, J=13.3, 6.6 Hz, 1H), 1.79-1.62 (m, 3H), 1.41-1.21 (m, 12H), 0.96 (d, J 6.5 Hz, 3H), 0.91 (d, J=6.7 Hz, 3H), 0.85 (td, J=6.7 Hz, 2.3, 3H), 0.80 (t, J=7.2 Hz, 3H). HRMS +ve calcd 369.2331, found 369.2332; −ve calcd 293.1787, found 293.1787.

### Cinnarizine Oleate

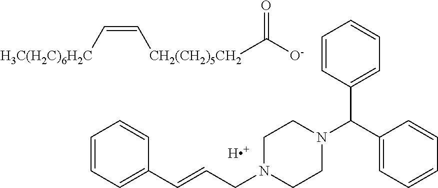

Method #3 has been used to make cinnarizine oleate.

¹H NMR (CDCl₃, 400 MHz) δ 9.83 (br s, 1H), 7.42-7.15 (m, 15H), 6.56 (d, J=15.8 Hz, 1H), 6.29 (dt, J=15.7, 7.1 Hz, 1H), 5.39-5.31 (m, 2H), 4.26 (s, 1H), 3.36 (d, J=7.0 Hz, 2H), 2.76 (br s, 4H), 2.54 (br s, 4H), 2.29 (t, J=7.6 Hz, 2H), 2.02 (m, J=6.5 Hz, 4H), 1.61 (quin, J=7.2 Hz, 2H), 1.32-1.27 (m, 20H), 0.89 (t, J=6.9 Hz, 3H). HRMS +ve calcd 369.2331, found 369.2333; −ve calcd 281.2481, found 281.2487.

### Cinnarizine Triflimide

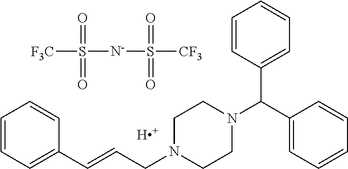

Method #3 has been used to make cinnarizine triflimide.

¹H NMR (CDCl₃, 400 MHz) δ7.43-7.19 (m, 15H), 6.79 (d, J=15.8 Hz, 1H), 6.22 (dt, J=15.5, 7.5 Hz, 1H), 4.32 (s, 1H), 3.87 (d, J=7.5 Hz, 2H), 3.51 (br s, 2H), 3.01 (br s, 4H), 2.48 (br s, 2H). HRMS +ve calcd 369.2331, found 369.2333; −ve calcd 279.9173, found 279.9184.

### Cinnarizine Stearate

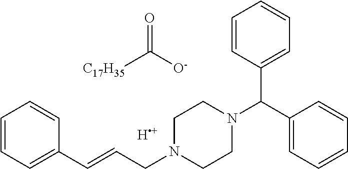

Method #2 has been used to make cinnarizine stearate.

¹H NMR (CDCl₃, 400 MHz) δ 8.55 (br s, 1H) 7.41-7.17 (m, 15H), 6.57 (d, J=15.8 Hz, 1H), 6.30 (dt, J=16.0, 7.2 Hz, 1H), 4.27 (s, 1H), 3.41 (d, J=7.0 Hz, 2H), 2.81 (br s, 4H), 2.58 (br s, 4H), 2.29 (t, J=7.6 Hz, 2H), 1.62 (quin, J=7.6 Hz, 2H), 1.32-1.26 (br s, 28H), 0.88 (t, J=6.8 Hz, 3H). HRMS +ve calcd 369.2331, found 369.2333; −ve calcd 283.2637, found 283.2635.

### Halofantrine Dodecylsulfate

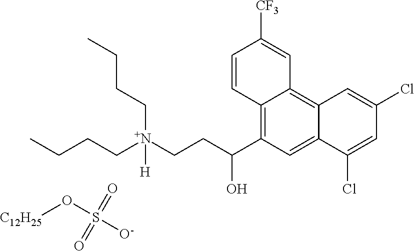

Method #3 has been used to make halofantrine dodecylsulfate.

¹H NMR (CDCl₃, 400 MHz) δ 8.53 (s, 1H), 8.25 (s, 1H), 8.23 (d, J=1.5 Hz, 1H), 8.12 (d, J=8.7 Hz, 1H), 7.74 (dd, J=8.7, 1.2 Hz, 1H), 7.53 (d, J=1.8 Hz, 1H) 5.64 (dd, J=8.9, 1.9 Hz, 1H), 3.97 (t, J=6.9 Hz, 2H), 3.44 (t, J=6.7 Hz, 2H), 3.05 (t, J=8.2 Hz, 4H), 2.32-2.28 (m, 1H), 2.14-2.04 (m, 1H), 1.72-1.64 (m, 4H), 1.58-1.51 (m, 2H), 1.40-1.27 (m, 22H), 0.93 (t, J=7.3 Hz, 6H), 0.87 (t, J=6.9 Hz, 3H), (—OH and —NH not observed). HRMS +ve calcd 500.1735, found 500.1740; −ve calcd 265.1474, found 265.1481.

### Halofantrine Oleate

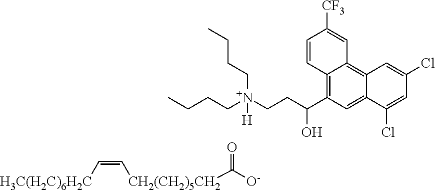

Method #3 has been used to make halofantrine oleate.

¹H NMR (CDCl₃, 400 MHz) δ8.83 (s, 1H), 8.54 (s, 1H), 8.52 (d, J=1.6 Hz, 1H), 8.23 (d, J=8.7 Hz, 1H), 7.84 (dd, J=8.7, 1.4 Hz, 1H), 7.71 (d, J=1.9 Hz, 1H), 5.69 (dd, J=8.4, 2.4 Hz, 1H), 5.38-5.29 (m, 2H), 3.02-2.88 (m, 2H), 2.78-2.70 (m, 2H), 2.64-2.56 (m, 2H), 2.30 (t, J=7.6 Hz, 2H), 2.21-2.14 (m, 1H), 2.07-1.98 (m, 5H), 1.64-1.56 (m, 6H), 1.43-1.26 (m, 24H), 0.97 (t, J=7.3 Hz, 6H), 0.88 (t, J=6.9 Hz, 3H), (—OH and —NH not observed). HRMS +ve calcd 500.1735, found 500.1741; −ve calcd 281.2481, found 281.2483.

### Halofantrine Triflimide

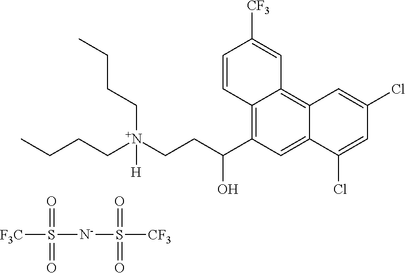 [SMILES: CCCC[NH+](CCCC)CCC(O)c1cc2c(Cl)cc(Cl)cc2c2cc(C(F)(F)F)ccc12 ; O=S(=O)([N-]S(=O)(=O)C(F)(F)F)C(F)(F)F]

Method #3 has been used to make halofantrine triflimide.

¹H NMR (CDCl₃, 400 MHz) δ8.42 (s, 1H), 8.10 (s, 1H), 8.08 (d, J=1.3 Hz, 1H), 7.86 (d, J=8.7 Hz, 1H), 7.71 (d, J=8.7 Hz, 1H), 7.41 (d, J=1.8 Hz, 1H), 5.63 (dd, J=8.0, 2.4 Hz, 1H), 3.50-3.43 (m, 1H), 3.34-3.28 (m, 1H), 3.14 (t, i=8.1 Hz, 4H), 2.31-2.26 (m, 1H), 2.01 (td, J=14.4, 7.9 Hz, 1H), 1.77-1.62 (m, 4H), 1.44-1.34 (m, 4H), 0.95 (t, J=7.3 Hz, 6H). HRMS +ve calcd 500.1735, found 500.1741; −ve calcd 279.9173, found 279.9183.

### Itraconazole Dodecylsulfate

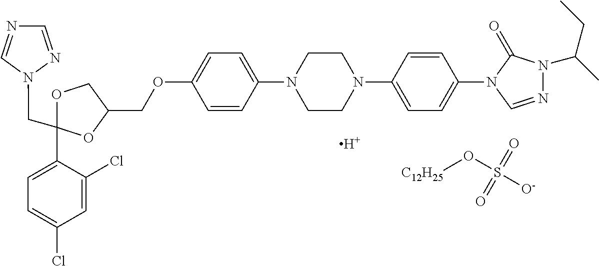

Modified method #3 (ethylacetate was used as a solvent instead of chloroform or dichloromethane) has been used to make itraconazole dodecysulfate

¹H NMR (CDCl₃, 400 MHz) δ 8.42 (s, 1H), 7.99 (s, 1H), 7.66 (overlapping d and s, 3H), 7.61 (d, J=8.4 Hz, 1H), 7.50 (overlapping doublets, 3H), 7.29 (dd, J=8.4, 2.1 Hz, 1H), 7.10 (d, J=8.9 Hz, 2H), 6.95 (d, J=9.1 Hz, 2H), 4.83 (q, J=14.8 Hz, 2H), 4.42-4.36 (m, 1H), 4.33-4.25 (m, 1H), 4.09 (t, J=6.8 Hz, 2H), 3.93 (dd, J=8.4, 6.8 Hz, 1H), 3.86-3.77 (m, 6H), 3.69-3.61 (m, 5H), 1.92-1.81 (m, 1H), 1.77-1.71 (m, 1H), 1.69-1.63 (m, 2H), 1.39 (d, J=6.7 Hz, 3H), 1.36-1.22 (m, 18H), 0.88 (overlap 2×t, 6H). HRMS +ve calcd 705.2471, found 705.2438; −ve calcd 265.1474, found 265.1480.

### Itraconazole 7-ethyl-2-methyl-4-undecyl sulfate

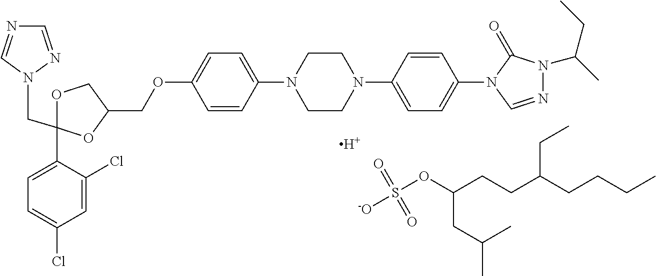

Modified Method #3 (ethylacetate was used as a solvent instead of chloroform or dichloromethane) has been used to make itraconazole 7-ethyl-2-methyl-4-undecyl sulfate.

¹H NMR (CDCl₃, 400 MHz) δ 8.25 (s, 1H), 7.92 (s, 1H), 7.64 (s, 1H), 7.59 (d, J=8.4 Hz, 1H), 7.55 (broad s, 2H), 7.49 (overlap 2×d, 3H), 7.28 (dd, J=8.4, 2.1 Hz, 1H), 7.08 (d, J=8.2 Hz, 2H), 6.92 (d, J=9.1 Hz, 2H), 4.81 (q, J=14.8 Hz, 21H), 4.55-4.49 (m, 1H), 4.41-4.35 (m, 1H), 4.34-4.25 (m, 1H), 3.93 (dd, J=8.4, 6.8 Hz, 1H), 3.84-3.74 (m, 6H), 3.60-3.53 (m, 5H), 1.91-1.78 (m, 2H), 1.76-1.61 (m, 4H), 1.40 (d, J=6.7 Hz, 3H), 1.37-1.19 (m, 12H), 0.93-0.83 (m, 12H), 0.79 (t, J=6.8 Hz, 3H). HRMS +ve calcd 705.2471, found 705.2466; −ve calcd 293.1787, found 293.1792.

### Itraconazole Dioctylsulfosuccinate (Docusate)

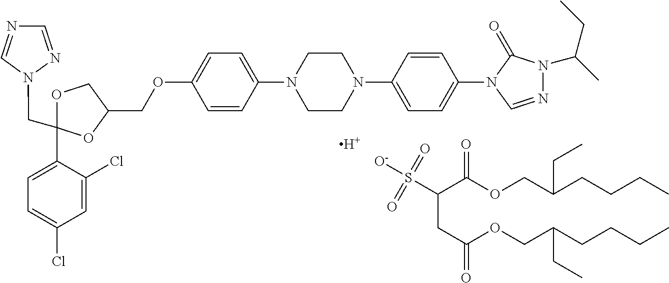

Modified Method #3 (ethylacetate was used as a solvent instead of chloroform or dichloromethane) has been used to make itraconazole docusate.

¹H NMR (CDCl₃, 400 MHz) δ 8.33 (s, 1H), 7.96 (s, 1H), 7.65 (s, 1H), 7.60 (2×d, 3H), 7.51 (d, J=8.9 Hz, 2H), 7.49 (d, J=2.1 Hz, 1H), 7.28 (dd, J=8.4, 2.1 Hz, 1H), 7.15 (d, J=7.2 Hz, 2H), 6.95 (d, J=9.1 Hz, 2H), 4.82 (q, J=14.8 Hz, 2H), 4.41-4.36 (m, 1H), 4.34-4.24 (m, 2H), 4.03-3.90 (m, 6H), 3.85-3.78 (m, 6H), 3.66-3.58 (m, 5H), 3.31-3.15 (m, 2H), 1.92-1.81 (m, 1H), 1.77-1.67 (m, 1H), 1.61-1.49 (m, 2H), 1.39 (d, J=6.7 Hz, 3H), 1.37-1.19 (m, 16H), 0.92-0.80 (m, 15H). HRMS +ve calcd 705.2471, found 705.2457; −ve calcd 421.2260, found 421.2258.

### Itraconazole Decahydronaphthalenylsulfate

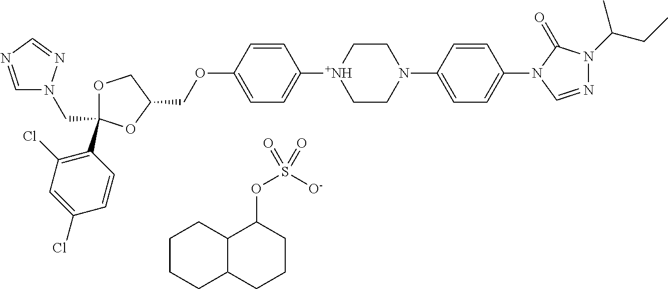 [SMILES: CCC(C)n1ncn(-c2ccc(N3CC[NH+](c4ccc(OC[C@H]5CO[C@](Cn6cncn6)(c6ccc(Cl)cc6Cl)O5)cc4)CC3)cc2)c1=O ; O=S(=O)([O-])OC1CCCC2CCCCC21]

Method #3 was used to make itraconazole decahydronaphthalenylsulfate

¹H NMR (d⁶-DMSO, 400 MHz) δ 8.43 (s, 1H), 8.34 (d, J=0.4 Hz, 1H), 7.88 (s, 1H) 7.69 (d, J=2.0 Hz, 1H), 7.50-7.54 (m, 3H), 7.43 (dd, J=8.4, 2.0 Hz, 1H), 7.12-7.22 (m, 4H), 6.94 (d, J=8.8 Hz, 2H), 4.88-4.75 (m, 2H), 4.37 (quin, J=6.0 Hz, 1H), 4.18-4.06 (m, 1H), 4.01 (dt, J=11.6, 4.6 Hz, 1H), 3.93 (dd, J=8.4, 6.8 Hz, 2H), 3.81-3.71 (m, 2H), 3.29-3.46 (m, 8H), 2.00-1.92 (m, 1H), 1.79-1.04 (m, 20H), 0.80 (t, J=7.4 Hz, 3H).

HRMS +ve mode: calcd. for C₃₅H₃₉Cl₂N₈O₄⁺ 705.2471 found 705.2443. HRMS −ve mode: calcd. for C₁₀H₁₇O₄S⁻ 233.0848 found 233.0852.

### Fexofenadine Dodecylsulfate

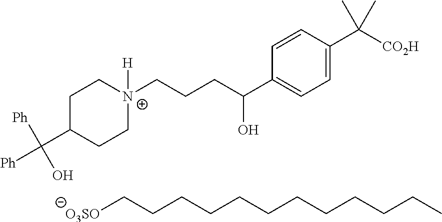

Method #2 was used to make Fexofenadine dodecylsulfate

¹H NMR (d₆-DMSO, 400 MHz) δ 12.29 (s, 1H), 8.83 (s, 1H), 7.48-7.50 (m, 4H), 7.26-7.31 (m, 8H), 7.16 (tt, J=7.6, 1.8 Hz, 2H), 5.64 (s, 1H), 5.28 (s, 1H), 4.51-4.54 (m, 1H), 3.66 (t, J=6.8 Hz, 2H), 3.38-3.46 (m, 2H), 2.78-3.01 (m, 5H), 1.55-1.73 (m, 6H), 1.43-1.48 (m, 10H), 1.22-1.29 (m, 18H), 0.85 (t, J=6.8 Hz, 3H), HRMS ⁺ve mode: calcd. for C₃₂H₄NO₄⁺ 502.2952 found 502.2959. HRMS ⁻ve mode: calcd. for C₁₂H₂₅O₄S⁻ 265.1479 found 265.1487.

### Fexofenadine Octadecylsulfate

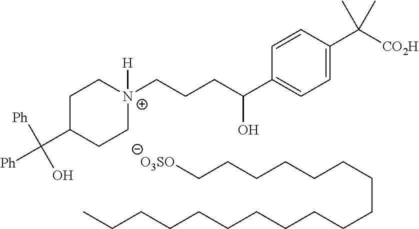

Method #2 was used to make Fexofenadine octadecylsulfate

¹H NMR (d₆-DMSO, 400 MHz) δ 12.27 (s, 1H), 8.83 (s, 1H), 7.48-7.50 (m, 4H), 7.26-7.31 (m, 8H), 7.16 (tt, J=7.2, 1.2 Hz, 2H), 5.64 (s, 1H), 5.28 (s, 1H), 4.51-4.54 (m, 1H), 3.66 (t, J=6.8 Hz, 2H), 3.38-3.46 (m, 2H), 2.79-3.02 (m, 5H), 1.57-1.70 (m, 6H), 1.43-1.49 (m, 10H), 1.21-1.29 (m, 30H), 0.85 (t, J=6.8 Hz, 3H), HRMS +ve mode: calcd. for C₃₂H₄₀NO₄⁺ 502.2952 found 502.2957. HRMS −ve mode: calcd. for C₁₈H₃₇O₄S⁻ 349.2418 found 349.2435.

### Fexofenadine Dioctylsulfosuccinate (Docusate)

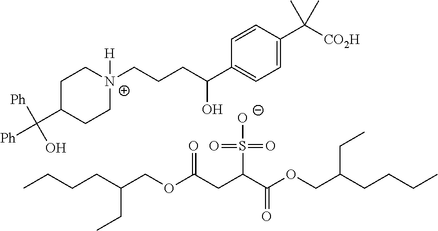 [SMILES: CC(C)(C(=O)O)c1ccc(C(O)CCC[NH+]2CCC(C(O)(c3ccccc3)c3ccccc3)CC2)cc1 ; CCCCC(CC)COC(=O)CC(C(=O)OCC(CC)CCCC)S(=O)(=O)[O-]]

Method #2 was used to make Fexofenadine dioctylsulfosuccinate

¹H NMR (d₆-DMSO, 400 MHz) δ 12.28 (s, 1H), 8.83 (s, 1H), 7.48-7.50 (m, 4H), 7.26-7.31 (m, 8H), 7.16 (tt, J=7.2, 1.2 Hz, 2H), 5.64 (s, 1H), 5.28 (s, 1H), 4.51-4.54 (m, 1H), 3.83-3.93 (m, 4H), 3.61 (dd, J=11.6, 3.6 Hz, 1H), 3.38-3.46 (m, 2H), 2.75-3.02 (m, 7H), 1.57-1.70 (m, 6H), 1.43-1.49 (m, 10H), 1.21-1.36 (m, 16H), 0.80-0.88 (m, 12H), HRMS ⁺ve mode: calcd. for C₃₂H₄₀NO₄⁺ 502.2952 found 502.2959. HRMS ⁻ve mode: calcd. for C₂₀H₃₇O₇S⁻ 421.2277 found 421.2265.

### Fexofenadine Decylsulfate

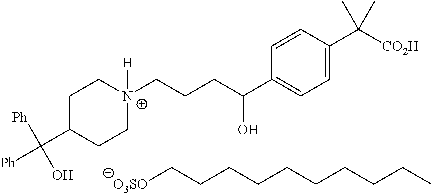

Method #2 was used to make Fexofenadine decylsulfate

¹H NMR (d₆-DMSO, 400 MHz) δ 12.29 (s, 1H), 8.83 (s, 1H), 7.48-7.50 (m, 4H), 7.26-731 (m, 8H), 7.16 (tt, J=7.2, 1.2 Hz, 2H), 5.64 (s, 1H), 5.28 (s, 1H), 4.51-4.54 (m, 1H), 3.66 (t, J=6.8 Hz, 2H), 3.38-3.46 (m, 2H), 2.78-3.01 (m, 5H), 1.55-1.73 (m, 6H), 1.43-1.48 (m, 10H), 1.22-1.29 (m, 14H), 0.85 (t, J=6.8 Hz, 3H), HRMS ⁺ve mode: calcd. for C₃₂H₄₀NO₄⁺ 502.2952 found 502.2959. HRMS ⁻ve mode: calcd. for C₁₀H₂₁O₄S⁻ 237.1166 found 237.1176.

### Fexofenadine 7-Ethyl-2-methyl-4-undecyl sulfate

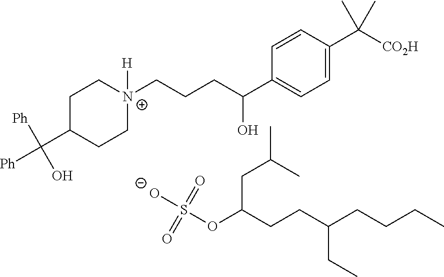 [SMILES: CC(C)(C(=O)O)c1ccc(C(O)CCC[NH+]2CCC(C(O)(c3ccccc3)c3ccccc3)CC2)cc1 ; CCCCC(CC)CCC(CC(C)C)OS(=O)(=O)[O-]]

Method #2 was used to make Fexofenadine 7-Ethyl-2-methyl-4-undecyl sulfate

¹H NMR (d₆-DMSO, 400 MHz) δ 12.28 (s, 1H), 8.83 (s, 1H), 7.48-7.50 (m, 4H), 7.26-7.31 (m, 8H), 7.14-7.18 (m, 2H), 5.62 (s, 1H), 5.28 (s, 1H), 4.51-4.53 (m, 1H), 4.03-4.09 (m, 1H), 3.39-3.52 (m, 4H), 3.38-3.46 (m, 2H), 2.75-3.03 (m, 5H), 1.36-1.76 (m, 18H), 1.14-1.29 (m, 10H), 0.78-0.88 (m, 12H), HRMS ⁺ve mode: calcd. for C₃₂H₄₀NO₄⁺ 502.2952 found 502.2960. HRMS ve mode: calcd. for C₁₄H₂₉O₄S⁻ 293.1792 found 237.1804.

### Fexofenadine Oleate

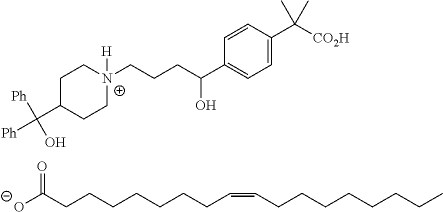 [SMILES: CC(C)(C(=O)O)c1ccc(C(O)CCC[NH+]2CCC(C(O)(c3ccccc3)c3ccccc3)CC2)cc1 ; CCCCCCCC/C=C\CCCCCCCC(=O)[O-]]

Method #3 was used to make Fexofenadine oleate

¹H NMR (d₆-DMSO, 400 MHz) δ 12.29 (s, 1H), 8.32 (s, 1H), 7.49-7.51 (m, 4H), 7.22-7.27 (m, 8H), 7.09-7.13 (m, 2H), 5.28-5.36 (m, 2H), 5.22 (s, 1H), 4.45 (t, J=6.0 Hz, 1H), 2.77-2.85 (m, 2H), 2.41-2.48 (m, 2H), 2.15-2.22 (m, 4H), 1.93-2.04 (m, 4H), 1.80-1.88 (m, 2H), 1.39-1.59 (m, 13H), 1.19-1.38 (m, 23), 0.85 (t, J=7.2 Hz, 3H), HRMS ⁺ve mode: calcd. for C₃₂H₄₀NO₄⁺ 502.2952 found 502.2960. HRMS ⁻ve mode: calcd. for C₁₈H₃₃O₂⁻ 281.2486 found 281.2484.

### Fexofenadine Octylsulfonate

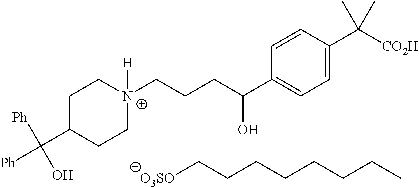

Method #2 was used to make Fexofenadine octylsulfonate

¹H NMR (d₆-DMSO, 400 MHz) δ 12.29 (s, 1H), 8.85 (s, 1H), 7.48-7.50 (m, 4H), 7.27-7.31 (m, 8H), 7.14-7.18 (m, 2H), 5.63 (s, 1H), 5.28 (s, 1H), 4.49-4.55 (m, 1H), 3.36-3.46 (m, 2H), 2.77-3.01 (m, 5H), 2.33-2.37 (m, 2H), 1.41-1.75 (m, 16H), 1.20-1.31 (m, 10H), 0.86 (t, J=6.8 Hz, 3H). HRMS ⁺ve mode: calcd. for C₃₂H₄₀NO₄⁺ 502.2952 found 502.2958.

HRMS ⁻ve mode: calcd. for C₈H₁₇O₃S⁻ 193.0904 found 193.0914.

### Fexofenadine Undecyltetrazolate

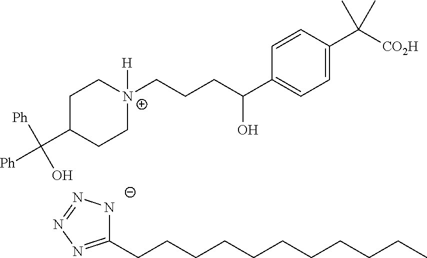 [SMILES: CC(C)(C(=O)O)c1ccc(C(O)CCC[NH+]2CCC(C(O)(c3ccccc3)c3ccccc3)CC2)cc1 ; CCCCCCCCCCCc1nnn[nH]1]

Method #3 was used to make Fexofenadine undecyltetrazolate.

¹H NMR (d⁴-MeOH, 400 MHz) δ 7.49-7.52 (m, 4H), 7.36-7.39 (m, 2H), 7.37 (dt. J=8.4, 2.0 Hz, 2H), 7.27-7.31 (m, 6H), 7.15-7.20 (m, 2H), 4.66 (t, J=6.2 Hz, 1H), 3.43-3.50 (m, 2H), 2.89-3.02 (m, 4H), 2.78-2.86 (m, 3H), 1.65-1.83 (m, 10H), 1.51 (s, 6H), 1.29-1.34 (17H), 0.88 (t, J=6.8 Hz, 3H), HRMS ⁺ve mode: calcd. for C₃₂H₄₀NO₄⁺ 502.2952 found 502.2959. HRMS ⁻ve mode: calcd. for C₁₂H₂₃N₄⁻ 223.1928 found 223.1937.

### Fexofenadine 3,7-dimethyloctanesulfate

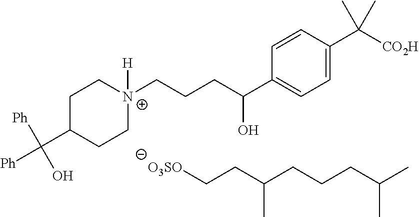

Method #2 was used to make Fexofenadine 3,7-dimethyloctanesulphate

¹H NMR (d₆-DMSO, 400 MHz) δ 12.26 (s, 1H), 8.83 (s, 1H), 7.48-7.50 (m, 4H), 7.26-7.31 (m, 8H), 7.14-7.18 (m, 2H), 5.63 (s, 1H), 5.27 (s, 1H), 4.51-4.54 (m, 1H), 3.66-3.75 (m, 21H), 3.36-3.47 (m, 2H), 2.76-3.05 (m, 4H), 1.40-1.74 (m, 17H), 1.04-1.32 (m, 8H), 0.82-0.86 (m, 9H), HRMS ⁺ve mode: calcd. for C₃₂H₄₀NO₄⁺ 502.2952 found 502.2956, HRMS ve mode: calcd. for C₁₀H₂₁O₄S⁻ 237.1166 found 237.1175.

### Fexofenadine Nonylsulfate

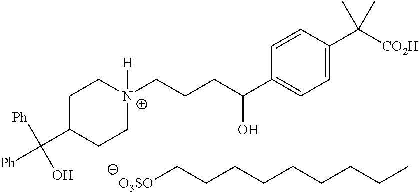

Method #2 was used to make Fexofenadine nonylsulfate

¹H NMR (d-DMSO, 400 MHz) δ 12.25 (s, 1H), 8.83 (s, 1H), 7.48-7.50 (m, 4H), 7.26-7.31 (m, 8H), 7.14-7.18 (m, 2H), 5.63 (s, 1H), 5.28 (s, 1H), 4.52 (t, J=6.0 Hz, 1H), 3.66 (t, J=6.8 Hz, 2H), 3.36-3.48 (m, 2H), 2.77-3.04 (m, 5H), 1.55-1.74 (m, 6H), 1.40-1.49 (m, 10H), 1.20-1.30 (m, 12H), 0.86 (t, J=6.8 Hz, 3H), HRMS ⁺ve mode: calcd. for C₃₂H₄₀NO₄⁺ 502.2952 found 502.2959. HRMS ⁻ve mode: calcd. for C₉H₁₉O₄S⁻ 223.1010 found 223.1015.

### Fexofenadine Dodecylsulfate/Fexofenadine Octylsulfate [1:1 Mix of Anions]

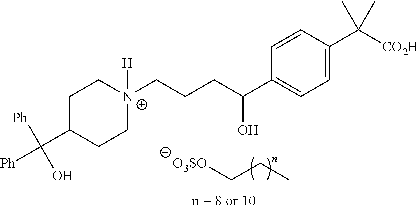

Method #2 (modified with 1:1 mixture of anions) was used to make Fexofenadine octyl/dodecylsulfate.

¹H NMR (d₆-DMSO, 400 MHz) δ 12.25 (s, 1H), 8.84 (s, 1H), 7.47-7.52 (m, 4H), 7.26-7.31 (m, 8H), 7.14-7.18 (m, 2H), 5.63 (s, 1H), 5.28 (s, 1H), 4.50-4.55 (m, 1H), 3.66 (t, J=6.4 Hz, 2H), 3.36-3.47 (m, 2H), 2.76-3.05 (m, 4H), 1.55-1.73 (m, 6H), 1.40-1.50 (m, 10H), 1.20-1.30 (m, 14H), 0.86 (t, J=6.8 Hz, 3H), HRMS ⁺ve mode: calcd, for C₃₂H₄₀NO₄⁺ 502.2952 found 502.2957. HRMS ⁻ve mode: calcd. for C₈H₁₇O₄S⁻ 209.0853 found 208.0863, calcd. for C₁₂H₂₅O₄S⁻ 265.1479 found 265.1491.

### Fexofenadine Adamantylsulfate

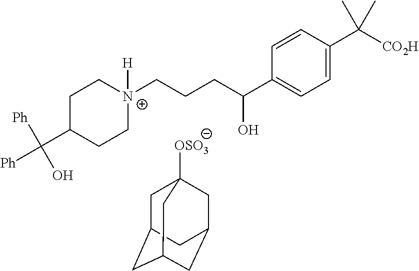

Method #3 was used to make Fexofenadine2-adamantylsulfate

¹H NMR (d₆-DMSO, 400 MHz) δ 12.22 (s, 1H), 8.83 (s, 1H), 7.48-7.50 (m, 4H), 7.26-7.31 (m, 8H), 7.14-7.18 (m, 2H), 5.64 (s, 1H), 5.28 (d, J=4 Hz, 1H), 4.51-4.54 (m, 1H), 4.31 (s, 1H), 3.39-3.46 (m, 2H), 3.37 (s, 2H), 2.78-3.01 (m, 4H), 2.00-2.05 (m, 3H), 1.43-1.73 (m, 24H), HRMS ⁺ve mode: calcd. for C₃₂H₄₀NO₄⁺ 502.2952 found 502.2957. HRMS ve mode: calcd. for C₁₀H₁₅O₄S⁻ 239.0697 found 239.0700.

### Dextromethorphan Decylsulfate (Mixture of Isomers [Cation])

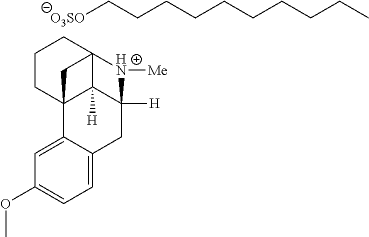

Method #2 was used to make dextromethorphan decylsulfate.

¹H NMR (d₆-DMSO, 400 MHz) (major) δ 9.46 (s, 1H), 7.12-7.15 (m, 1H), 6.81-6.84 (m, 2H), 3.73 (s, 3H), 3.66 (t, J=6.8 Hz, 2H), 3.60-3.62 (m, 1H), 3.11-3.22 (m, 2H), 2.93-3.01 (m, 2H), 2.83 (d, J=4.8 Hz, 2H), 2.36-2.47 (m, 2H), 1.91 (dt, J=12.4, 2.4 Hz, 1H), 1.74 (dt, J=13.6, 4.4 Hz, 1H), 1.58-1.65 (m, 1H), 1.41-1.53 (m, 5H), 1.20-1.40 (m, 17H), 1.11-1.19 (m, 1H), 0.92-1.01 (m, 1H), 0.85 (t, J=6.8 Hz, 3H).

¹H NMR (d₆-DMSO, 400 MHz) (minor) δ 9.46 (s, 1H), 7.12-7.15 (m, 1H), 6.81-6.84 (m, 2H), 3.73 (s, 3H), 3.66 (t, J=6.8 Hz, 2H), 3.53-3.57 (m, 1H), 3.11-3.22 (m, 2H), 2.93-3.01 (m, 2H), 2.83 (d, J=4.8 Hz, 2H), 2.36-2.47 (m, 2H), 2.17-2.22 (m, 1H), 2.04 (dt, J=14.0, 4.4 Hz, 1H), 1.58-1.65 (m, 1H), 1.41-1.53 (m, 5H), 1.20-1.40 (m, 17H), 1.11-1.19 (m, 1H), 0.92-1.01 (m, 1H), 0.85 (t, J=6.8 Hz, 3H).

HRMS ⁺ve mode: calcd. for C₁₈H₂₆NO⁺ 272.2009 found 272.2010. HRMS ⁻ve mode: calcd. for C₁₀H₂₁O₄S⁻ 237.1166 found 237.1172.

### Dextromethorphan Dodecylsulphate (Mixture of Isomers)

Method #2 was used to make dextromethorphan dodecylsulfate

(major) ¹H NMR (d⁶-DMSO, 400 MHz) (major) δ 9.46 (s, 1H), 7.12-7.15 (m, 1H), 6.81-6.84 (m, 2H), 3.73 (s, 3H), 3.66 (t, J=6.8 Hz, 2H), 3.60-3.62 (m, 1H), 3.11-3.22 (m, 2H), 2.93-3.01 (m, 2H), 2.83 (d, J=4.8 Hz, 3H), 2.36-2.47 (m, 2H), 1.91 (dt, J=12.4, 2.4 Hz, 1H), 1.74 (dt, J=13.6, 4.4 Hz, 1H), 1.58-1.65 (m, 1H), 1.41-1.53 (m, 5H), 1.20-1.40 (m, 20H), 1.11-1.19 (m, 1H), 0.92-1.01 (m, 1H), 0.85 (t, J=6.8 Hz, 3H).

(minor) ¹H NMR (d⁶-DMSO, 400 MHz) (minor) δ 9.46 (s, 1H), 7.12-7.15 (m, 1H), 6.81-6.84 (m, 2H), 3.73 (s, 3H), 3.66 (t, J=6.8 Hz, 2H), 3.53-3.57 (m, 1H), 3.11-3.22 (m, 2H), 2.93-3.01 (m, 2H), 2.95 (d, J=4.8 Hz, 3H), 2.36-2.47 (m, 2H), 2.17-2.22 (m, 1H), 2.04 (dt, J=14.0, 4.4 Hz, 18), 1.58-1.65 (m, 1H), 1.41-1.53 (m, 5H), 1.20-1.40 (m, 20H), 1.11-1.19 (m, 1H), 0.92-1.01 (m, 1H), 0.85 (t, J=6.8 Hz, 3H).

### Metformin Octylsulfonate

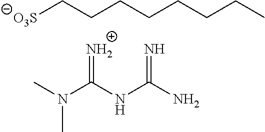

Method #3 was used to make Metformin octylsulfonate.

¹H NMR (d₆-DMSO, 400 MHz) δ 7.18 (s, 2H), 6.64 (s, 4H), 2.92 (s, 6H), 2.36-2.40 (m, 2H), 1.50-1.58 (m, 2H), 1.20-1.31 (m, 10H), 0.85 (t, J=7.2 Hz, 3H), HRMS ⁺ve mode: calcd. for C₄H₁₂N₅⁺ 130.1087 found 130.1090 (2.16 ppm). HRMS ⁻ve mode: calcd. for C₈H₁₇O₃S⁻ 193.0904 found 193.0909 (2.51 ppm).

### Metformin Dodecylsulfate

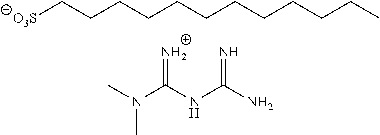

¹H NMR (d₆-DMSO, 400 MHz) δ 6.34-7.25 (m, 6H), 3.66 (t, J=6.8 Hz, 2H), 2.92 (s, 6H), 1.47 (quin, J=6.8 Hz, 2H), 1.20-1.30 (m, 18H), 0.85 (t, J=6.8 Hz, 3H), HRMS ⁺ve mode: calcd. for C₄H₁₂N₅⁺ 130.1087 found 130.1091. HRMS ⁻ve mode: calcd. for C₁₂H₂₅O₄S⁻ 265.1479 found 265.1491.

### Methods for Acidic Drugs

### Method #4

Developed for acidic drugs.
Used particularly for water-soluble counter ions such as alkylpyridinium salts and quaternary ammonium salts.
Drugs: Applicable to acidic drugs such as ibuprofen, diclofenac, meclofenamic and tolfenamic acid
Metathesis reactions using acidic drugs should ideally be carried out under basic condition by addition of alkali salts (e.g. sodium carbonate, sodium bicarbonate etc.)
Metathesis reactions using acidic drugs should ideally be carried out in water and methanol when using highly lipophilic counterions which are insoluble in water.
Metathesis reactions using acidic drugs should ideally be carried out by adding strong base (NaOH, KOH etc.) when using free acids instead of acidic drug salts as a starting material.

### Example Application: Meclofenamic Acid, N-octyl-3-methylpyridinium Salt

Sodium carbonate was added to distilled water (20 mL) and the pH was adjusted to 9-10. Meclofenamic acid sodium salt (84.7 mg, 0.27 mmol) was dissolved in 10 mL of this aq. basic solution. N-octyl-3-mnethylpyridinium bromide (83.8 mg, 0.29 mmol) was also dissolved in 10 mL of this aq, basic solution. The two solutions were mixed and oil droplets were immediately formed in the water phase. The reaction mixture was stirred further for 30 min. The oil phase was extracted with DCM (3×20 mL) from water. The combined DCM phases were washed with distilled water (4×30 mL) until a negative AgNO₃ test was obtained. The organic phase was then dried (anhydrous MgSO₄), filtered and evaporated to afford the desired product, which was dried at 60° C. under high vacuum. Yield 97%.

### Method #5

### Example Application: Ibuprofen Octylammonium Salt

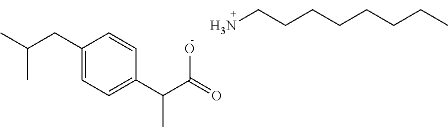

To ibuprofen (75.2 mg, 0.37 mmol) solution in acetonitrile (2 mL) was added n-octyl amine (47.1 mg, 0.37 mmol) solution in acetonitrile (2 mL). A white precipitate was formed and was washed with acetonitrile prior to filtration under vacuum. Yield 95%.

¹H NMR (DMSO-d₆, 400 MHz) δ 7.16 (d, J=8.0 Hz, 2H), 7.01 (d, J=8.1 Hz, 2H), 3.37 (q, J=7.1 Hz, 1H), 2.59 (t, J=7.2 Hz, 2H), 2.38 (d, J=7.1 Hz, 2H), 1.78 (sept, J=6.8 Hz, 1H), 1.43-1.37 (m, 2H), 1.29-1.23 (m, 13H), 0.86 (2×t, 9H), ¹³C NMR (CDCl₃, 100 MHz) δ 181.9, 141.4, 139.4, 129.1, 127.2, 48.6, 45.2, 39.4, 32.0, 30.3, 29.4, 29.3, 28.2, 26.8, 22.8, 22.6, 19.7, 14.2 HRMS +ve calcd 130.1.596 found 130.1599: −ve calcd 205.1229 found 205.1227.

### Ibuprofen Dodecylammonium Salt

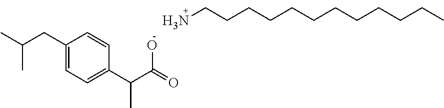

Modified Method #5 (where acetonitrile and methanol were used as solvents) was used to make ibuprofen dodecylammonium salt.

¹H NMR (DMSO-d₆, 400 MHz) δ 7.16 (d, J=7.9 Hz, 2H), 6.97 (d, J=7.9 Hz, 2H), 3.37 (q, J=7.1 Hz, 1H), 2.59 (t, J=7.6 Hz, 2H), 2.36 (d, J=7.1 Hz, 2H), 1.77 (sept, J=6.7 Hz, 1H), 1.44-1.41 (m, 2H), 1.28 (d, J=7.1 Hz, 6H), 1.22 (br s, 15H), 0.84 (2×t, 9H). HRMS +ve calcd 186.2222, found 186.2222 −ve calcd 205.1229, found 205.1226.

### Tolfenamic Acid, Butylammonium Salt

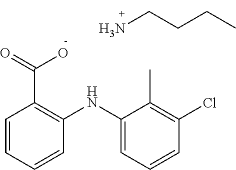

Modified Method #5 (where methanol was used as a solvent) was used to make Tolfenamic acid, butylammonium salt.

¹H NMR (DMSO-d₆, 400 MHz) δ 8.38 (br s), 7.92 (dd, J=7.7, 1.7 Hz, 1H), 7.31 (dd, J=8.1, 0.7 Hz, 1H), 7.18-7.14 (m, 1H), 7.11 (t, J 8.0 Hz, 1H), 7.04 (dd, J=8.2, 0.8 Hz, 1H), 6.99 (dd, J=7.9, 0.7 Hz, 1H), 6.69 (m, 1H), 2.79 (t, J=8.0 Hz, 2H), 2.29 (s, 3H), 1.58-1.51 (m, 2H), 1.37-1.28 (m, 2H), 0.86 (t, J=7.4 Hz, 3H), HRMS −ve calcd 260.0478 found 260.0489.

### Tolfenamic Acid, Octylammonium Salt

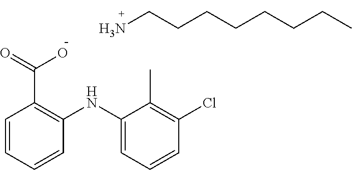

Modified Method #5 (where methanol was used as a solvent) was used to make tolfenamic acid, octylammonium salt.

¹H NMR (DMSO-d₆, 400 MHz) δ 8.15 (br s), 7.90 (dd, J=7.7, 1.7 Hz, 1H), 7.30 (dd, J=8.1, 0.7 Hz, 1H), 7.17-7.13 (m, 1H), 7.10 (t, J=8.0 Hz, 1H), 7.03 (dd, J=8.2, 0.8 Hz, 1H), 6.97 (dd, J=7.9, 0.7 Hz, 1H), 6.69-6.65 (m, 1H), 2.77 (t, J=8.0 Hz, 2H), 2.29 (s, 3H), 1.58-1.50 (m, 2H), 1.30-1.22 (m, 10H), 0.84 (t, J=6.8 Hz, 3H). HRMS +ve calcd 130.1596, found 130.1598; −ve calcd 260.0478, found 260.0491.

### Tolfenamic Acid, Dodecylammonium Salt

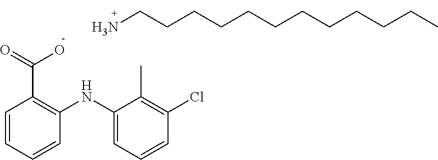 [SMILES: CCCCCCCCCCCCN ; Cc1c(Cl)cccc1Nc1ccccc1C(=O)O]

Modified Method #5 (where acetonitrile and methanol were used as solvents) was used to make tolfenamic acid, dodecylammonium salt.

¹H NMR (DMSO-d₆, 400 MHz) δ 7.89 (dd, J=7.7, 1.7 Hz, 1H), 7.30 (dd, J=8.1, 0.7 Hz, 1H), 7.16-7.08 (m, 2H), 7.03 (dd, J=8.2, 0.9 Hz, 1H), 6.96 (dd, J=7.9, 0.6 Hz, 1H), 6.68-6.64 (m, 1H), 2.76 (t, J=7.4 Hz, 2H), 2.28 (s, 3H), 1.56-1.48 (m, 2H), 1.27-1.22 (m, 18H), 0.85 (t, J=6.8 Hz, 3H), HRMS +ve calcd 186.2222, found 186.2222; −ve calcd 260.0478, found 260.0486.

### Tolfenamic Acid, N-butyl-N,N-dimethylbutyl-N-dodecylammonium Salt

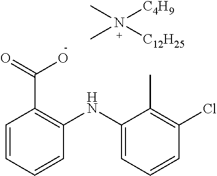

Modified Method #4 (where 2M NaOH added into the reaction mixture) was used to make tolfenamic acid, N-butyl-N,N-dimethylbutyl-N-dodecylammonium salt.

¹H NMR (DMSO-d₆, 400 MHz) δ 7.88 (dd, J=7.6, 1.6 Hz, 1H), 7.29 (d, J=7.9, 1H), 7.11-7.03 (m, 3H), 6.91 (d, J=7.8 Hz, 1H), 6.66-6.62 (m, 1H), 3.24-3.19 (m, 4H), 2.98 (s. 6H), 2.32 (s, 3H), 1.63-1.57 (m, 4H), 1.33-1.24 (m, 20H), 0.92 (t, J=7.3 Hz, 3H, 0.85 (t, J=6.8 Hz, 3H), HRMS +ve calcd 270.3161, found 270.316 −ve calcd 260.0478, found 260.0491.

### Tolfenamic Acid, N-decyl-N,N-dimethyldodecylammonium Salt

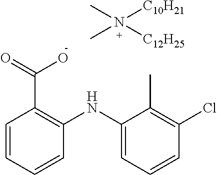

Modified Method #4 (where 2M NaOH was added into the reaction mixture and methanol and water were used as solvents) was used to make tolfenamic acid, N-decyl-N,N-dimethyldodecylammonium salt.

¹H NMR (CDCl₃, 400 MHz) δ 8.07 (dd, J=7.7, 1.5 Hz, 1H), 7.31 (dd, J=7.8, 1.0 Hz, 1H), 7.16-7.07 (m, 2H), 7.01-6.93 (m, 2H), 6.72-6.68 (m, 1H), 3.31-3.26 (m and s overlapping, 10H), 2.37 (s, 3H), 1.56 (br s, 4H), 1.22 (br s, 32H), 0.87 (2×t, 6H), HRMS +ve calcd 354.41 found 354.41 −ve calcd 260.0478 found 260.049.

### Meclofenamic Acid, 1-octyl-3-methylpyridinium Salt

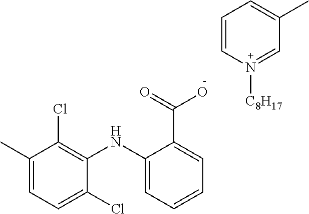

Method #4 was used to make meclofenamic acid, 1-octyl-3-methylpyridinium salt

¹H NMR (DMSO-d₆, 400 MHz) δ 9.03 (s, 1H), 8.94 (d, J=6.0 Hz, 1H), 8.43 (d, J=8.0 Hz, 1H), 8.05 (dd, J=7.9, 6.1 Hz, 1H), 7.82 (dd, J=7.6, 1.7 Hz, 1H), 7.40 (d, J=8.2 Hz, 1H), 7.18 (dd, J=8.3, 0.6 Hz, 1H), 6.97-6.93 (m, 1H), 6.53 (td, J=7.5, 1.1 Hz, 1H), 6.01 (dd, J=8.1, 0.9 Hz, 1H), 4.54 (t, J=7.6 Hz, 2H), 2.5 (s, 3H), 2.35 (s, 3H), 1.94-1.87 (m, 2H), 1.26-1.23 (m, 10H), 0.85 (t, J=6.9 Hz, 3H), HRMS +ve calcd 206.1909, found 206.1908; −ve calcd 294.0089, found 294.0102.

### Meclofenamic Acid, 1-hexadecyl-3-methylpyridinium Salt

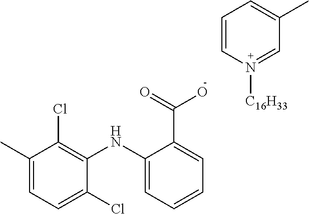

Modified Method #4 (methanol and water used as solvents) was used to make meclofenamic acid, 1-hexadecyl-3-methylpyridinium salt.

¹H NMR (DMSO-d₆, 400 MHz) δ 9.05 (s, 1H), 8.95 (d, J=6.0 Hz, 1H), 8.43 (d, J=8.0 Hz, 1H), 8.04 (dd, J=7.9, 6.1 Hz, 1H), 7.82 (dd, J=7.6, 1.7 Hz, 1H), 7.39 (d, J=8.2 Hz 1H), 7.17 (dd, J=8.3, 0.5 Hz, 1H), 6.96-6.92 (m, 1H), 6.53 (td, J=7.5, 1.1 Hz, 1H), 6.00 (dd, J=8.1, 0.9 Hz, 1H), 4.54 (t, J=7.6 Hz, 2H), 2.50 (s, 3H), 2.35 (s, 3H), 1.93-1.88 (m, 2H), 1.28-1.23 (m, 26H), 0.85 (t, J=6.8 Hz, 3H), HRMS +ve calcd 318.3161, found 318.3162; −ve calcd 294.0089, found 294.0098.

### Meclofenamic Acid, N-butyl-N,N-dimethyldodecylammonium Salt

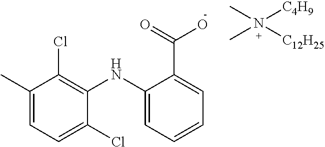

Method #4 was used to make meclofenamic acid, N-butyl-N,N-dimethyldodecylammonium salt.

¹H NMR (DMSO-d₆, 400 MHz) δ 7.81 (dd, J=7.6, 1.7 Hz, 1H), 7.40 (d, 1=8.2 Hz, 1H), 7.17 (d, J=8.3, 1H), 6.96-6.92 (m, 1H), 6.53 (td, J=7.5, 1.1 Hz, 1H), 6.00 (dd, J=8.1, 0.9 Hz, 1H), 3.24-3.19 (m, 4H), 2.98 (s, 6H), 2.35 (s, 3H), 1.67-1.58 (m, 41H), 1.34-1.24 (m, 20H), 0.92 (t, J=7.4 Hz, 3H), 0.85 (t, J=6.8 Hz, 3H), HRMS +ve calcd 270.3161, found 270.316; −ve calcd 294.0089, found 294.0096.

### Meclofenamic Acid, N-decyl-N,N-dimethyldodecylammonium Salt

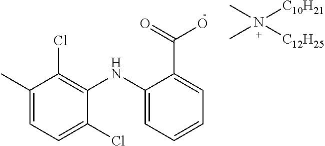

Modified Method #4 (where methanol and water were used as solvents) was used to make meclofenamic acid, N-decyl-N N-dimethyldodecylammonium salt

¹H NMR (DMSO-d₆, 400 MHz) δ 7.81 (dd, J=7.6, 1.6 Hz, 1H), 7.40 (d, J=8.2 Hz, 1H), 7.17 (d, J=8.3, 1H), 6.96-6.92 (m, 1H), 6.53 (td, J=7.6, 1.0 Hz, 1H), 6.00 (dd, J=8.1 Hz, 1H), 3.23-3.19 (m, 4H), 2.98 (s, 6H), 2.35 (s, 3H), 1.66-1.60 (m, 4H), 1.24 (br s, 32H), 0.85 (2×t, 6H), HRMS +ve calcd 354.41, found 354.41 −ve calcd 294.0089, found 294.0097.

### Diclofenac, 1-octyl-3-mnethylpyridinium Salt

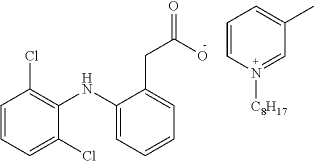

Method #4 was used to make diclofenac Diclofenac, 1-octyl-3-methylpyridinium salt.

¹H NMR (CDCl₃, 400 MHz) δ10.50 (br s, 1H), 9.03 (s, 1H), 8.94 (d, J=6.0 Hz, 1H), 8.43 (d, J=8.0 Hz, 1H), 8.04 (dd, J=7.9, 6.2 Hz, 1H), 7.43 (d, J=8.0 Hz, 2H), 7.06-7.00 (t and dd overlapping, 2H), 6.90 (td, J=7.7, 1.6 Hz, 1H), 6.70 (td, J=7.4, 1.1 Hz, 1H), 6.21 (dd, J=7.9, 0.9 Hz, 1H), 4.53 (t, J=7.5 Hz, 2H), 3.35 (s, 2H), 2.49 (s, 3H), 1.93-1.88 (m, 2H), 1.26-1.23 (m, 10H), 0.85 (t, J=6.9 Hz, 3H), HRMS +ve calcd 206.1909, found 206.1911; −ve calcd 294.0089, found 294.0102.

### Diclofenac, N-alkyl-N-benzyl-N,N-dimethylammonium Salt

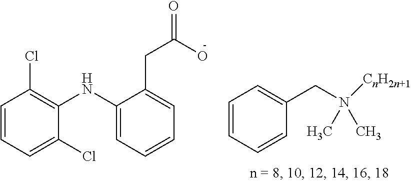

Modified Method #4 (where methanol and water were used as solvents) was used to make diclofenac, N-alkyl-N-benzyl-N,N-dimethylammonium salt.

¹H NMR (CDCl₃, 400 MHz) δ 9.22 (br s, 1H), 7.47-7.36 (m, 6H), 7.27 (d, J 6.8 Hz, 1H), 7.21 (d, J=6.9 Hz, 1H), 6.94 (td, J=7.8, 1.3 Hz, 1H), 6.88 (t, J=8.0 Hz, 1H), 6.76 (t, J=7.1 Hz, 1H), 6.43 (d, J=7.6 Hz, 1H), 4.70 (s, 2H), 3.76 (s, 2H), 3.20 (t, J=7.6 Hz, 2H), 3.04 (s, 6H), 1.62 (m, 2H), 1.31-1.22 (m, 23H), 0.88 (t, J=6.8 Hz, 3H), HRMS +ve calcd 304.3004, found 304.3004 and calcd 332.3317 found 332.3319; −ve calcd 294.0089, found 294.0099.

### Valsartan, N-decylpyridinium Salt

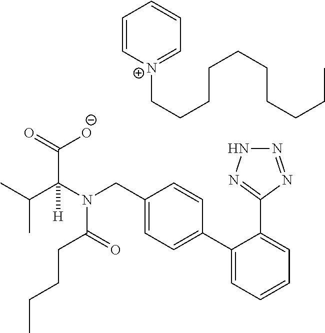

Method #4 was used to make valsartan, N-decylpyridinium salt,

¹H NMR (d₆-DMSO, 400 MHz) (major) δ 9.10-9.14 (m, 2H), 8.55-8.61 (m, 1H), 8.10-8.14 (m, 2H), 6.94-7.75 (m, 8H), 4.57 (t, J=7.6 Hz, 2H), 4.31-4.80 (m, 2H), 3.70-3.84 (m, 1H), 2.47-2.54 (m, 1H), 2.35-2.43 (m, 1H), 1.72-2.17 (m, 3H), 1.03-1.55 (m, 19H), 0.83-0.92 (m, 8H), 0.5-0.74 (m, 4H).

¹H NMR (d₆-DMSO, 400 MHz) (minor) δ 9.10-9.14 (m, 2H), 8.55-8.61 (m, 1H), 8.10-8.14 (m, 2H), 6.94-7.75 (m, 8H), 4.57 (t, J=7.6 Hz, 2H), 4.31-4.80 (m, 2H), 3.70-3.84 (m, 1H), 1.72-2.17 (m, 4H), 1.03-1.55 (m, 19H), 0.83-0.92 (m, 8H), 0.5-0.74 (m, 4H).

HRMS ⁺ve mode: calcd, for C₁₅H₂₆N⁺ 220.2060 found 220.2059. HRMS ⁻ve mode: calcd. for C₂₄H₂₈N₅O₃⁻ 424.2198 found 424.2216.

### Valsartan, N-hexadecyl-N,N,N-trimethylammonium Salt

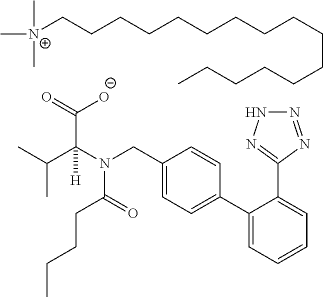 [SMILES: CCCCCCCCCCCCCCCC[N+](C)(C)C ; CCCCC(=O)N(Cc1ccc(-c2ccccc2-c2nn[nH]n2)cc1)[C@H](C(=O)[O-])C(C)C]

Method #4 was used to make Valsartan, N-hexadecyl-N,N,N-trimethylammonium salt.

¹H NMR (d₆-DMSO, 400 MHz) (major) δ 7.48-7.53 (m, 1H), 7.27-7.38 (m, 3H), 6.92-7.12 (m, 4H), 4.60 (d, J=15.0 Hz, 1H), 4.41 (d, J=15.0 Hz, 1H), 3.65 (d, J=10.4 Hz, 1H), 3.23-3.27 (m, 2H), 3.00 (s, 9H), 2.48-2.56 (m, 1H), 2.33-2.41 (m, 1H), 1.93-2.06 (m, 1H), 1.21-1.67 (m, 36H), 1.09 (sext, J=7.6 Hz, 2H), 0.62-0.90 (m, 12H).

¹H NMR (d₆-DMSO, 400 MHz) (minor) δ 7.48-7.53 (m, 1H), 7.27-7.38 (m, 31H), 6.92-7.12 (m, 4H), 4.90 (d, J=17.0 Hz, 1H), 4.56 (d, J=10.4 Hz, 1H), 4.38 (d, J=17.0 Hz, 1H), 3.23-3.27 (m, 21H), 3.00 (s, 91H), 2.08-2.15 (m, 1H), 1.93-2.06 (m, 1H), 1.79-1.86 (m, 1H), 1.21-1.67 (m, 36H), 1.09 (sext, J=7.6 Hz, 2H), 0.62-0.90 (m, 12H).

HRMS ⁺ve mode: calcd. for C₁₉H₄₂N⁺ 284.3312 found 284.3313. HRMS ⁻ve mode: calcd. for C₂₄H₂₈N₅O₃⁻ 424.2198 found 424.2201.

### Melting Point and Solubility Data for Low Melting Ionic Salts

Tables 1-10 summarise melting point suppression data for a range of low melting ionic salts. Exemplar lipid formulations have also been constructed and the maximum drug solubility in that formulation measured to provide an indication of the possible advantages in solubility that are possible due to low melting ionic salt formation. Where lipid based formulations have been employed, formulations were made up in glass vials by weighing the appropriate quantities of excipient directly into the vial, followed by mixing

The following formulations were constructed to exemplify the utility of ionic salt formation in increasing solubility in lipid based formulations. They are typical of contemporary lipid based formulations that spontaneously self emulsify on contact with gastrointestinal fluids—often called self emulsifying drug delivery systems (SEDDS), and typically comprise mixtures of lipids, surfactants and a cosolvent.

LC¹ SEDDS: 15% w/w soybean oil (SBO), 15% w/w Maisine, 60% w/w Cremophor EL (CrEL), 10% w/w EtOH

LC² SEDDS: 30% w/w SBO, 30% w/w Maisine, 30% CrEL w/w, 10% w/w EtOH

MCSEDDS: 15% w/w Captex 355, 15% w/w Capmul MCM, 60% w/w CrEL, 1.0% w/w EtOH.

In some cases the solubility of the low melting ionic salt in individual excipients was also measured.

Drug solubility in each formulation was assessed in one of two ways. Firstly, quantitatively, by incubating formulations with excess drug at 37 degrees and taking samples over time. These samples were centrifuged to pellet solid material and the drug concentration in the formulation assessed by HPLC. Equilibrium solubility was assumed to have been reached when solubility values in successive samples varied by less than 10%.

Where solubilities were very high and essentially miscible values are shown as >X where X is the upper limit that was tested.

In other cases a visual solubility was obtained by incubating formulations with a known quantity of drug and then adding additional drug where the first quantity of drug passed into solution over a 12 hr period. Where a solubility limit is not reached values are shown as >X where X is the upper limit that was shown to be soluble.

Where melting points and melting ranges are provided, in some cases these might more accurately be referred to as glass transition state temperatures, especially for those ionic salts with melting points approaching room temperature.

TABLE 1
Cinnarizine

| | | | Solubility | Solubility |
|---|---|---|---|---|
| | | | in LC¹ | in MC¹ |
| | | | SEDDS | SEDDS |
| | | Melting | (mg/g) | (mg/g) |
| | | point | cinnarizine | cinnarizine |
| Drug/Drug-ILs | Counterion | (° C.) | equiv. | equiv. |
| 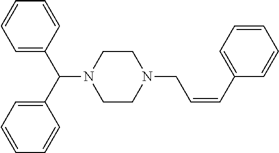 [SMILES: C(=C/c1ccccc1)/CN1CCN(C(c2ccccc2)c2ccccc2)CC1] Cinnadzine free base | — | 118-120 | 43.6 | 43.2 |
| Cinnarizine-decylsulfate* | 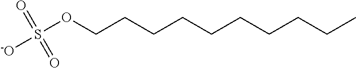 | viscous oil | >300 | >330 |
| Cinnarizine-lauryl(docecyl)sulfate | 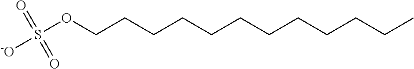 | viscous oil | >230 | >220 |
| Cinnarizine-octadecylsulfate | 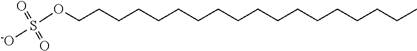 | 78-81 | 52.6 | 63.2 |
| Cinnarizine-7-ethyl-2- methyl-4-undecyl sulfate | 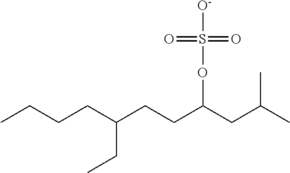 [SMILES: CCCCC(CC)CCC(CC(C)C)OS(=O)(=O)[O-]] | viscous oil | >240 | >260 |
| Cinnarizine-oleate | 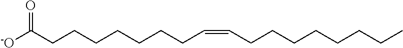 | 93-98 | 82.8 | 93.7 |
| Cinnarizine-stearate | 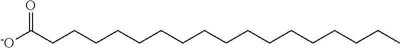 | 79-83 | 72.8 | 80.6 |
| Cinnarizine-triflimide | 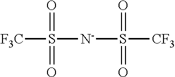 [SMILES: O=S(=O)([N-]S(=O)(=O)C(F)(F)F)C(F)(F)F] | 38-43 | >320 | >300 |

*The solubility of cinnarizine decylsulphate IL was also measured in individual excipients and was >249 mg/g in Captex 355, >289 mg/g in Capmul MCM and >119 mg/g in Cremophor EL, Similar to the data in the formulations this is significantly higher than that for cinnarizine FB in the equivalent excipients

TABLE 2
Halofantrine

| | | | Solubility | Solubility |
|---|---|---|---|---|
| | | | in LC¹ | in MC¹ |
| | | | SEDDS | SEDDS |
| | | Melting | (mg/g) | (mg/g) |
| | | point | halofantrine | halofantrine |
| Drug/Drug-ILs | Counterion | (° C.) | equiv. | equiv. |
| 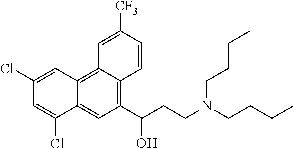 [SMILES: CCCCN(CCCC)CCC(O)c1cc2c(Cl)cc(Cl)cc2c2cc(C(F)(F)F)ccc12] Halofantrine free base | — | 79-82 | 76.8 | 74.9 |
| Halofantrine- dodecylsulfate | 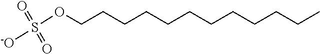 | 78-81 | 95.8 | 140.5 |
| Halofantrine-oleate | 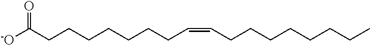 | liquid | >330 | >320 |
| Halofantrine- triflimide | 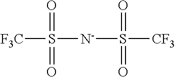 [SMILES: O=S(=O)([N-]S(=O)(=O)C(F)(F)F)C(F)(F)F] | viscous oil | >350 | >340 |

TABLE 3
Itraconazole

| | | | Solu- | Solu- |
|---|---|---|---|---|
| | | | bility | bility |
| | | | in LC¹ | in MC¹ |
| | | | SEDDS | SEDDS |
| | | Melt- | (mg/g) | (mg/g) |
| | | ing | itracon- | itracon- |
| | | point | azole | azole |
| Drug/Drug-ILs | Counterion | (° C.) | equiv. | equiv. |
| 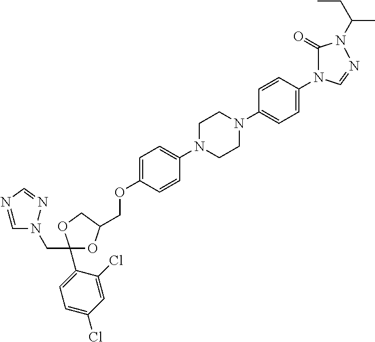 [SMILES: CCC(C)n1ncn(-c2ccc(N3CCN(c4ccc(OCC5COC(Cn6cncn6)(c6ccc(Cl)cc6Cl)O5)cc4)CC3)cc2)c1=O] Itraconazole free base | — | 170 | 2.2 | 2.7 |
| Itraconazole-HCl | Cl | | 16.4 | 20.2 |
| Itraconazole- dodecylsulfate | 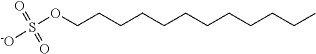 | 145- 150 | 23.3 | 25.7 |
| Itraconazole-7-ethyl-2- methyl-4-undecyl sulfate | 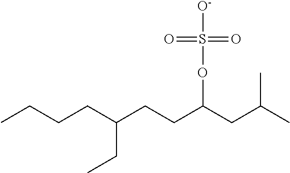 [SMILES: CCCCC(CC)CCC(CC(C)C)OS(=O)(=O)[O-]] | 53-60 | 75.4 | 75.7 |
| Itraconazole- diocytlsulfosuccinate | 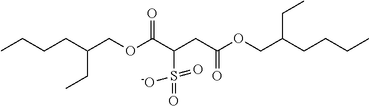 | 47-53 | 106.8 in LC¹ 159.8 in LC² | 115 |
| Itraconazole decahydronaphthalen- 1-yl sulphate | 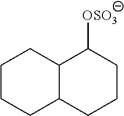 | 87- 109 | | |

TABLE 4
Fexofenadine

| | | | Solubility | Solubility |
|---|---|---|---|---|
| | | | in LC¹ | in MC |
| | | | SEDDS | SEDDS |
| | | Melting | (mg/g) | (mg/g) |
| | | point | itraconazole | itraconazole |
| Drug/Drug-ILs | Counterion | (° C.) | equiv. | equiv. |
| Fexofenadine | N/A | 142-143 | | |
| Fexofenadine HCl | Cl | 192-194 | | |
| Fexofenadine decylsulfate | 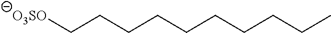 | 55-72 | <50 | <50 |
| Fexofenadine dodecylsulfate* | 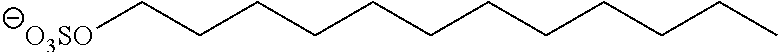 | 54-72 | | >250 |
| Fexofenadine octadecylsulfate | 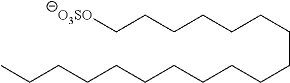 | 50-65 | | >250 |
| Fexofenadine 7-ethyl-2- methylundecyl-4- sulfate | 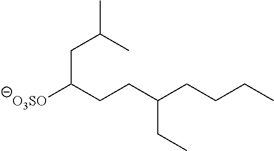 | 46-63 | | 100-200 |
| Fexofenadine docusate | 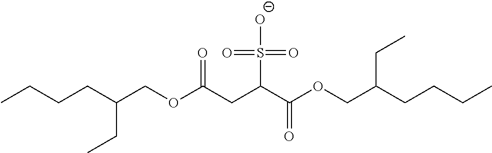 [SMILES: CCCCC(CC)COC(=O)CC(C(=O)OCC(CC)CCCC)S(=O)(=O)[O-]] | 45-70 | | >250 |
| Fexofenadine oleate | 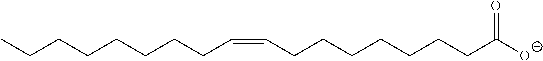 [SMILES: CCCCCCCC/C=C\CCCCCCCC(=O)[O-]] | 48-64 | | >250 |
| Fexofenadine 5- undecyltetrazolate | 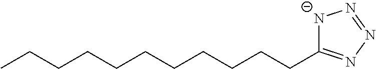 [SMILES: CCCCCCCCCCCc1nnn[n-]1] | 72-91 | | >100 |
| Fexofenadine 3,7-dimethyloctane sulfate | 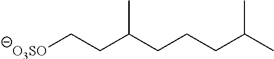 | 58-80 | | 100-200 |
| Fexofenadine nonylsulfate | 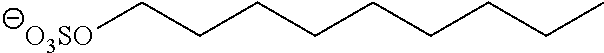 | 59-81 | | >250 |
| Fexofenadine adamantylsulfate | 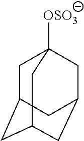 | 84-101 | | 100-200 |
| Fexofenadine octylsulfonate | 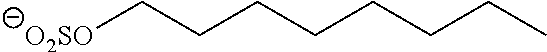 | 61-84 | | >250 |

The solubility of fexofenadine dodecyl sulphate was also evaluated in a prototype formulation comprising 40% w/w Kolliphor RH 40, 40% w/w Labrasol (PEG-8 Caprylic/Capric Glycerides) and 20% w/w Capryol 90 (Propylene glycol monocaprylate). The solubility in this formulation was >520 mg/g

TABLE 5
Dextromethorphan and Metformin

| | | | Solubility | Solubility |
|---|---|---|---|---|
| | | | in LC¹ | in MC |
| | | | SEDDS | SEDDS |
| | | Melting | (mg/g) | (mg/g) |
| Drug/Drug IL | | point | Free base | Free base |
| Counterion | Structure | (° C.) | equiv. | equiv. |
| Dextromethorphan | | 111 | | <50 |
| Dextromethorphan Decylsulfate | 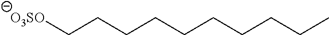 | 62-68 | | 50-100 |
| Dextromethorphan dodecylsulfate | 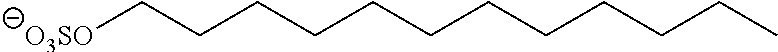 | oil | | |
| Metformin | | 222-226 | | |
| Metformin octylsulfonate | 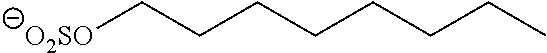 | 112-119 | | |
| Metformin dodecylsulfate | 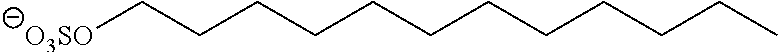 | 73-75 | | 10-25 |

TABLE 6
Ibuprofen

| | | | Solubility | Solubility |
|---|---|---|---|---|
| | | | in LC² | in MC² |
| | | | SEDDS | SEDDS |
| | | Melting | (mg/g) | (mg/g) |
| | | point | Free base | Free base |
| Drug/Drug IL | Counterion | (° C.) | equiv. | equiv. |
| 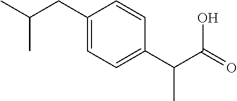 Ibuprofen | — | 73-76 lit 76-79 | | |
| Ibuprofen octylammonium salt | 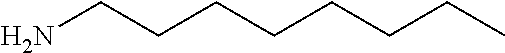 | 75-77 | | |
| Ibuprofen dodecylammonium salt | 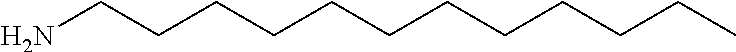 | 69-72 | | |

TABLE 7
Tolfenamic Acid

| | | | Solubility | Solubility |
|---|---|---|---|---|
| | | | in LC² | in MC² |
| | | | SEDDS | SEDDS |
| | | Melting | (mg/g) | (mg/g) |
| | | point | Free base | Free base |
| Drug/Drug IL | Counterion | (° C.) | equiv. | equiv. |
| 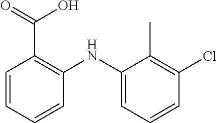 [SMILES: Cc1c(Cl)cccc1Nc1ccccc1C(=O)O] Tolfenamic acid | — | 213 | 25 < sol < 48 | 30 < sol < 50 |
| Tolfenamic acid, butylammonium salt | 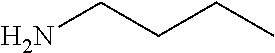 | 169-171 | | |
| Tolfenamic acid, octylammonium salt | 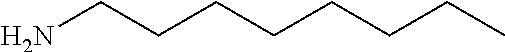 | 146-149 | | |
| Tolfenamic acid dodecylammonium salt | 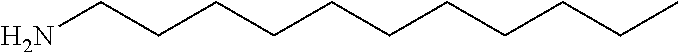 | 127-129 | >48 | >50 |
| Tolfenamic acid, N-butyl- N,N-dimethylbutyl-N- dodecylammonium salt | 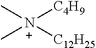 | 98-100 | >150 | >200 |
| Tolfenamic acid, N-decyl-N,N- dimethyldodecylammonium salt | 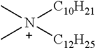 | 48-70 | >200 | >200 |

TABLE 8
Meclofenamic Acid

| | | | Solubility | Solubility |
|---|---|---|---|---|
| | | | in LC² | in MC² |
| | | | SEDDS | SEDDS |
| | | Melting | (mg/g) | (mg/g) |
| | | point | Free base | Free base |
| Drug/Drug IL | Counterion | (° C.) | equiv. | equiv. |
| 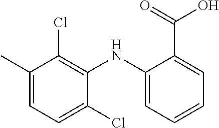 [SMILES: Cc1ccc(Cl)c(Nc2ccccc2C(=O)O)c1Cl] Meclofenamic acid | — | 257-259 | | |
| Meclofenamic acid, sodium salt | Na | 289-291 | 58 < sol < 84 | |
| Meclofenamic acid, 1-octyl-3- methylpyridinium salt | 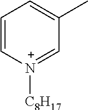 | oil | | |
| Meclofenamic acid, 1- hexadecyl-3- methylpyridinium salt | 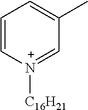 | oil | >240 | >240 |
| Meclofenamic acid, N-butyl- N,N-dimethyldodecylammonium salt | 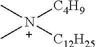 | 111-113 | >154 | >220 |
| Meclofenamic acid, N-decyl-N,N- dimethyldodecylammonium salt | 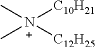 | 107-113 | >227 | >240 |

TABLE 9
Diclofenac

| | | | Solubility | Solubility |
|---|---|---|---|---|
| | | | in LC² | in MC² |
| | | | SEDDS | SEDDS |
| | | Melting | (mg/g) | (mg/g) |
| | | point | Free base | Free base |
| Drug/Drug IL | Counterion | (° C.) | equiv. | equiv. |
| 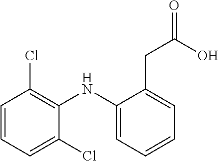 [SMILES: O=C(O)Cc1ccccc1Nc1c(Cl)cccc1Cl] Diclofenac | — | 180 | | |
| Diclofenac sodium salt | Na | 284 | 58 < sol < 84 | 115 < sol < 166 |
| Diclofenac, 1-octyl-3- methylpyridinium salt | 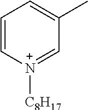 | oil | | |
| Diclofenac, N-alkyl-N- benzyl-N,N- dimethylammonium salt | 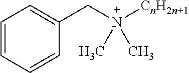 n = 8, 10, 12, 14, 16, 1 | oil | >238 | >238 |

TABLE 10
Valsartan

| | | | Solubility | Solubility |
|---|---|---|---|---|
| | | | in LC¹ | in MC |
| | | | SEDDS | SEDDS |
| | | Melting | (mg/g) | (mg/g) |
| | | point | Free base | Free base |
| Drug/Drug IL | Counterion | (° C.) | equiv. | equiv. |
| Valsartan | | 116-117 | | |
| Valsartan, N- decylpyridinium salt | 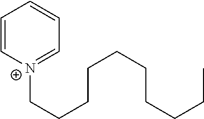 [SMILES: CCCCCCCCCC[n+]1ccccc1] 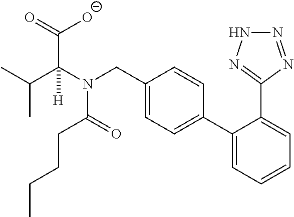 [SMILES: CCCCC(=O)N(Cc1ccc(-c2ccccc2-c2nn[nH]n2)cc1)[C@H](C(=O)[O-])C(C)C] | 68-87 | | |
| Valsartan, N- hexadecyl-N,N,N- trimethylammonium salt″ | 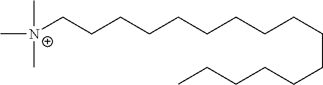 [SMILES: CCCCCCCCCCCCCCCC[N+](C)(C)C] 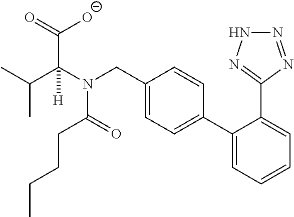 [SMILES: CCCCC(=O)N(Cc1ccc(-c2ccccc2-c2nn[nH]n2)cc1)[C@H](C(=O)[O-])C(C)C] | viscous oil | | |

### Example 2

### In Vivo Data for Comparative Formulations of Cinnarizine

Various formulations of cinnarizine free base (FB) and decylsulfate ionic liquid (IL) were prepared according to Table 2-1. As the free base, cinnarizine solubility in the lipid vehicle is approximately 44 mg/g. Formulations are rarely loaded with drug at 100% of their solubility in the lipid vehicle since this provides a risk of drug precipitation from the formulation if storage temperatures fluctuate etc., so typically, drugs might be loaded at about 80% of saturation. In this instance, this dictates a maximum loading of ˜35 mg/g. In contrast the decylsulphale IL of cinnarizine is essentially miscible with the formulation and could be loaded at almost any drug load. In this example the drug was loaded at either 35 mg/g to match that which could be achieved with the FB, and at ˜125 mg/g as an exemplar higher level that was achievable using the IL. Control formulations were also generated at 125 mg/g as an aqueous suspension of cinnarizine decylsulfate IL and at 125 mg/g as a suspension of the FB in the SEDDS formulation.

TABLE 2-1
Formulations of cinnarizine FB and IL

| Formulation | Dose | |
|---|---|---|
| Cinnarazine decylsulfate IL (SEDDS^{#} solution) | 125* | mg/g |
| Cinnarazine decylsulfate IL (SEDDS^{#} solution) | 35* | mg/g |
| Cinnarazine FB (SEDDS^{#} suspension) | 125* | mg/g |
| Cinnarazine FB (SEDDS^{#} solution) | 35* | mg/g |
| Cinnarazine decylsulfate IL (aqueous suspension) | 125* | mg/g |

*free base equivalents
^{#}15% w/w soybean oil, 15% w/w Maisine 35-1, 60% w/w Cremophor EL. 10% w/w EtOH. The combination of polar and non polar lipids along with a surfactant and co-solvent is used to help dispersion of the components in the gastrointestinal tract.

The SEDDS solution formulations were prepared as follows, although other methods may be used: the individual components of the lipid formulation were weighed directly into a glass vial before mixing and incubation until a single phase lipid vehicle was produced. Subsequently, the free base or decylsulfate salt of cinnarizine was weighed into a fresh glass vial, followed by the lipid vehicle, up to the target mass, and the mixture was stirred to form a single phase formulation.

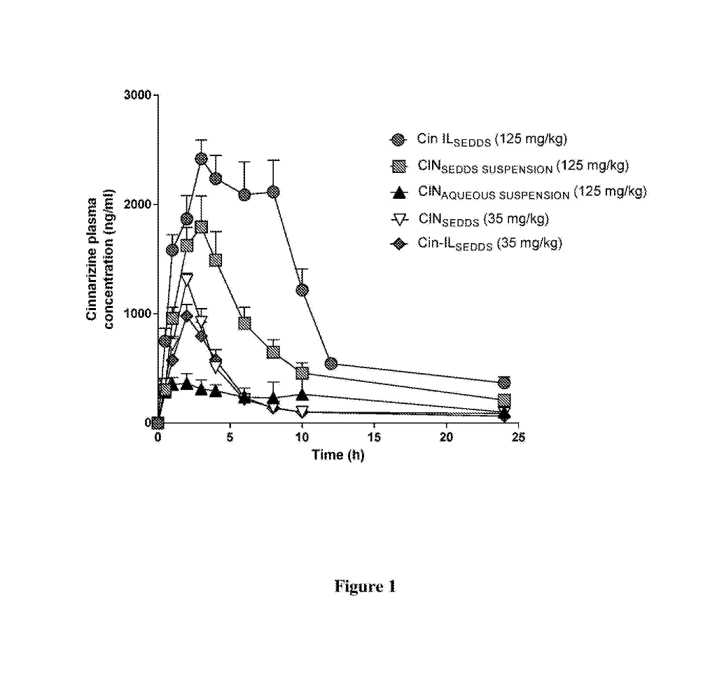

Formulations were administered to overnight fasted rats by oral gavage at a formulation dose of 1 mL/kg (˜280 mg formulation/rat) dispersed in 1 mL of water. Cinnarizine FB and cinnarizine IL were dosed as either a solution in a self emulsifying lipid based formulation (SEDDS), as a suspension in the same SEDDS or as an aqueous suspension formulation. Rats had cannulas inserted into the carotid artery to allow blood samples to be taken over time. The concentration of cinnarizine in plasma was then measured by HPLC-MS. The results are depicted in FIG. 1 and Table 2-2 below.

The data suggest that at the lower dose, where the SEDDS formulation was able to dissolve either Cin FB or Cin IL, Cin plasma exposure was similar and, as expected, higher than the aqueous suspension. Importantly, however the Cin IL allowed formulation into the SEDDS formulation as a solution at a much higher dose (125 mg·kg⁻¹), resulting in significantly higher exposure than the same dose of Cin FB in the same SEDDS formulation, since the lack of solubility of Cin FB dictated formulation as a suspension in the SEDDS rather than a solution (FIG. 1).

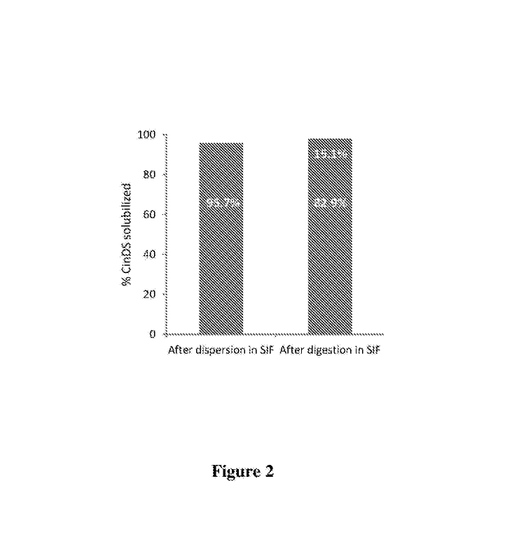

A key criteria for lipid based formulations such as SEDDS is that they maintain drug in a solubilised state as the formulation is dispersed in the fluids of the stomach and is subsequently digested on contact with lipase enzymes in the intestine. Thus FIG. 2 shows that the synthesis of the Cin IL not only allows for much greater quantites of Cin to be dissolved in a lipid based formulation, but that the IL remains solubilised in the formulation as it is dispersed and digested in the GI tract. After in vitro dispersion or digestion more than 95% of the incorporated CinDS remained solubilized in an aqueous phase (methods as Williams et at J. Pharm. Sci. (2012) 101, 3360-3380). After digestion, a small proportion of the solubilized CinDS was recovered in a phase separated oil phase. Effective continued solubilisation of Cin IL is consistent with the high absorption and systemic exposure seen in vivo.

TABLE 2-2
Pharmacokinetic parameters for cinnarizine free base (Cin FB) after
administration of either Cin FB or cinnarizine ionic liquid (Cin IL)

| | | Dose^{(a)} | | | |
|---|---|---|---|---|---|
| | | (mg | AUC_{0-24 h} | Cₘₐₓ | Tₘₐₓ |
| | | kg⁻¹) | (ng h ml⁻¹) | (ng ml⁻¹) | (h) |
| Cin IL | SEDDS | 125 | 26063 ± 2370 | 2629 ± 248 | 4.9 ± 0.8 |
| solution | | | | | |
| Cin FB | SEDDS | 125 | 14770 ± 1860 | 1800 ± 283 | 2.8 ± 0.3 |
| suspen- | | | | | |
| sion | | | | | |
| Cin FB | Aqueous | 125 | 5277 ± 2671 | 355.7 ± 80.0 | 2.0 ± 0.6 |
| suspen- | | | | | |
| sion | | | | | |
| Cin FB | SEDDS | 35 | 5844 ± 487 | 1305 ± 64.2 | 2.0 ± 0.0 |
| solution | | | | | |
| Cin IL | SEDDS | 35 | 5240 ± 494 | 916.2 ± 107 | 2.2 ± 0.2 |
| solution | | | | | |

^{(a)}Cinnarizine in free base equivalents.
In all cases, the SEDDS formulation consisted of 15% (w/w) soybean oil, 15% (w/w) Maisine 35-1 ™, 60% (w/w) Cremophor EL and 10% (w/w) ethanol.

### Example 3

### A Surfactant-Free Formulation Containing Cinnarizine Decylsulfate

A formulation (4 g) was prepared containing the following:

| | |
|---|---|
| Cinnarizine decylsulfate | 0.5 g |
| Medium-chain triglyceride (Migylol 812) | 3.5 g |

The alkylsulfate salt of cinnarizine was weighed into a fresh glass vial, followed by the medium-chain triglyceride up to the target mass. The IL salt of cinnarizine was incorporated into the formulation through overnight stirring at room temperature to form a single phase formulation.

### Example 4

### A Semi-Solid Lipid Formulation Containing Cinnarizine Decylsulfate

A formulation (4 g) was prepared containing the following:

| Cinnarizine decylsulfate | 0.5 g |
|---|---|
| PEG glycerides of lauric acid (Gelucire ® | 3.5 g |
| 44/14) | |

The decylsulfate salt of cinnarizine was weighed into a fresh glass vial, followed by pre-melted Gelucire® up to the target mass. The IL salt of cinnarizine was incorporated into the formulation through overnight stirring at elevated temperature to form clear solution, after which the formulation was cooled resulting in a single phase formulation that is solid/semi-solid at room temperature.

### Example 5

### In Vivo Data for Comparative Formulations of Itraconazole

Data similar to that generated for cinnarizine have been obtained for an aqueous suspension of itraconazole free base (ITZ FB), a suspension of itraconzole free base in a SEDDS formulation (LC² SEDDS: 30% w/w SBO, 30% w/w Maisine, 30% CrEL w/w, 10% w/w EtOH), a solution of itraconazole docusate IL (ITZ IL) in the same SEDDS formulation and the commercial spray dried dispersion formulation of ITZ (Sporanox). Note that the very low solubility of ITZ free base precludes formulation as a lipid based formulation. It is only via isolation as the IL that this possibility is realised since the solubility of the ITZ IL in lipid formulations is very much higher. All were dosed at the same dose (20 mg/kg). The formulation details are presented in Table 5-1.

TABLE 5-1
Formulations of itraconazole.

| | | | | | Nominal | Actual |
|---|---|---|---|---|---|---|
| | | Weight | | Dose Volume | Dose | Dose |
| Group | Rat | (g) | Formulation | (mL) | (mg/kg) | (mg/kg) |
| ITZ FB Aqueous | 1 | 265 | 6.25 mg ITZ FB in | 0.5 mL | 20.8 | 23.5 |
| Suspension | 2 | 267 | suspension vehicle | (Followed by | | 23.4 |
| | 3 | 251 | (0.5% w/v NaCMC, | 0.5 mL water) | | 24.9 |
| | 4 | 248 | 0.4% v/v Tween 80 and | | | 25.2 |
| | | | 0.9% NaCl in water) | | | |
| | | | | | Mean ± SD | 24.2 ± 0. [symbol] |
| ITZ FB SEDDS^{a} | 5 | 292 | 6.25 mg ITZ FB in | 0.5 mL | 20.8 | 21.4 |
| Suspension | 6 | 277 | SEDDS vehicle (30% | (Followed by | | 22.5 |
| | 7 | 286 | SBO, 30% Maisine, 30% | 0.5 mL water) | | 21.8 |
| | 8 | 295 | Cremophor EL, 10% | | | 21.2 |
| | | | ethanol) | | | |
| | | | | | Mean ± SD | 21.7 ± 0. [symbol] |
| Commercial | 9 | 245 | 6.25 mg ITZ FB (~29 mg | Neat | 20.8 | 25.5 |
| Sporanox ® | 10 | 283 | of capsule contents, | (Followed by | | 22.0 |
| Formulation | 11 | 267 | Janssen-Cilag Pty Ltd) | 0.5 mL water) | | 23.4 |
| | 12 | 276 | | | | 22.6 |
| | | | | | Mean ± SD | 23.4 ± 1. [symbol] |
| ITZ IL | 13 | 270 | 6.25 mg ITZ FB (~10 mg | 0.5 mL | 20.8 | 23.1 |
| Formulation | 14 | 294 | ITZ-Docusate IL) in | (Followed by | | 21.2 |
| | 15 | 295 | 1.25 mg SEDDS vehicle | 0.5 mL water) | | 21.2 |
| | 16 | 283 | (30% SBO, 30% | | | 22.0 |
| | | | Maisine, 30% | | | |
| | | | Cremophor EL, 10% | | | |
| | | | ethanol) in water | | | |
| | | | | | Mean ± SD | 21.9 ± 0. [symbol] |

^{a}SEDDS vehicle similar but not identical to SEDDS used to cinnarizine study. In this case SEDDS contains 30% w/w SBO, 30% w/w Maisine, 30% CrEL w/w, 10% EtOH [symbol]
indicates data missing or illegible when filed

The isolation of ITZ as the docusate IL increased drug solubility in the SEDDS formulation and allowed administration as a Solution in the SEDDS formulation. This resulted in significantly higher plasma levels (˜2.5 fold) when compared to the commercial formulation after administration of the same equivalent dose of ITZ FB. ITZ FB was not sufficiently soluble in the SEDDS formulation to allow administration as a solution in the SEDDS at any reasonable dose and was therefore dosed as a suspension in the SEDDS formulation and also as an aqueous suspension. The same dose was administered as the commercial Sporanox formulation of ITZ FB

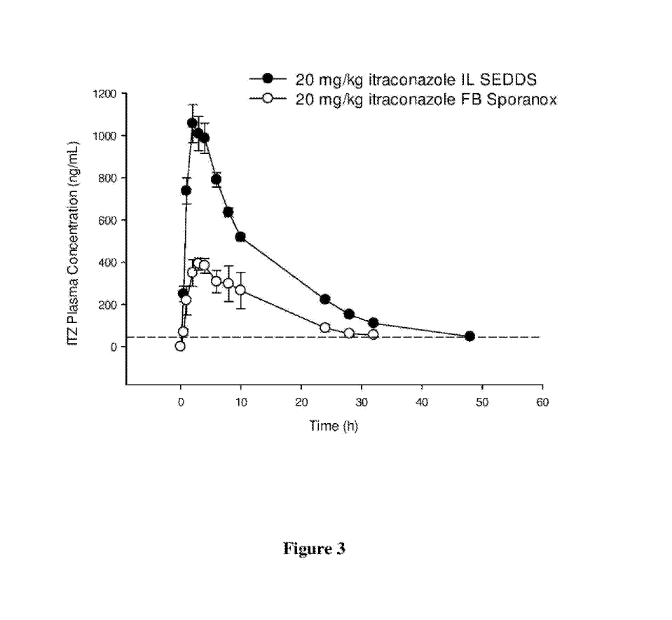

FIG. 3 shows that in vivo itraconazole exposure was extremely low after oral administration of the aqueous suspension of ITZ FB and the suspension of ITZ FB in the SEDDS formulation. In fact in both cases drug concentrations in plasma were below the limit of quantification of the assay (shown as the dotted line in FIG. 3). The current commercial oral formulation (Sporanox) led to moderate plasma levels.

Summary pharmacokinetic data for itraconazole plasma concentration versus time data after administration of the four comparative oral itraconazole formulations is given in Table 5-1. Data are shown for the administration of 20 mg/kg itraconazole either as the commercial reference formulation (Sporanox) or as itraconazole docusate (20 mg/kg itraconazole equivalents) dissolved in a lipid based formulation comprising (30% w/w soybean oil, 30% w/w Maisine 35-1, 30% w/w Cremophor EL, 10% w/w EtOH). Itraconazole was also dosed at 20 mg/kg as a suspension in the same lipid based formulation and also as an aqueous suspension. In both of the latter two cases plasma concentrations were below the limit of quantification of the assay (50 ng/mL) at all time points. The first time point for the itraconazole plasma level time curve was below the limit of quantification, but measurable peaks were apparent and data are included as an estimate. It is apparent that the ITZ IL formulation allowed administration of a much higher ITZ dose as a solution in a lipid based formulation and that this in turn led to much higher plasma levels than the equivalent suspension formulation of the free base, or the commercial Sporanox formulation.

TABLE 5-2
Pharmacokinetic parameters for itraconazole after oral
administration of itraconazole free base and itraconazole
docusate ionic liquid containing formulations

| | Tₘₐₓ | Cₘₐₓ | AUC₀₋ₗₐₛₜ | |
|---|---|---|---|---|
| | (h) | (ng/mL) | (ng/mL*h) | F % |
| ITZ FB Aqueous | — | <50 | <LOQ* | NA |
| Suspension | | | | |
| ITZ FB SEDDS | — | <50 | <LOQ | NA |
| Suspension | | | | |
| ITZ FB Sporanox | 3.8 ± 1.4 | 460 ± 31 | 5855 ± 1158 | 100 |
| ITZ-IL SEDDS solution | 2.8 ± 0.5 | 1065 ± 87 | 14420 ± 687 | 246 |

*F % provides the relative bioavailability of itraconazole when compared to the commercial formulation

As described above for cinnarizine, in addition to enhancing drug solubility in a lipid based formulations, the IL also increased drug solubility and affinity for colloidal species that are present in the gastrointestinal tract as a lipid based formulation is processed, digested and solubilised by intestinal fluids. Table 5.3 below shows the equilibrium solubility of ITZ FB and ITZ docusate in the colloids formed by in vitro digestion of the formulation used in the in vivo studies in FIG. 3. In this experiment blank SEDDS formulation (1 g) was dispersed in 39 mL of simulated intestinal fluid. (SIF) (2 mM Tris-maleate, 1.4 mM CaCl₂.H₂O, 150 mM NaCl, 3 mM NaTDC, 0.75 mM PC, pH 6.5, 37° C.) and pancreatic enzymes added to stimulate digestion. The experiment was conducted at 37° C. and allowed to continue for 60 mins. At the end of 60 mins digestion was stopped by the addition of an enzyme inhibitor and drug solubility in the colloids produced by digestion assessed. From the data in Table 5.3 below it is apparent that synthesis of the IL increases drug affinity for intestinal colloidal phases and therefore increases solubilisation in the GI tract—consistent with the increases in exposure seen in FIG. 3

TABLE 5.3
Solubility of ITZ docusate IL and ITZ free base in colloidal
species formed by digestion of lipid based formulations

| | Simulated intestinal |
|---|---|
| | fluid plus lipid |
| | degestion products |
| ITZ-docusate IL | 1720 ± 150 μg/mL |
| ITZ FB | <47 μg/mL |

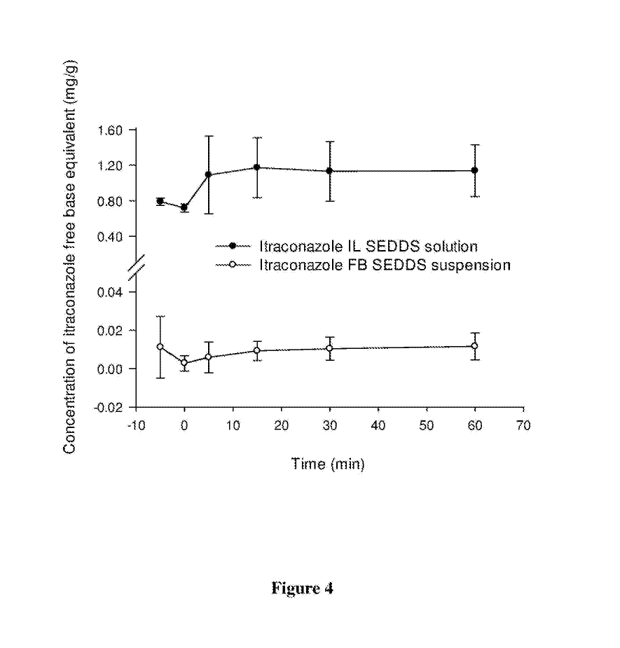

FIG. 4 also shows that after dissolving ITZ-IL in a lipid based formulation and assessing behaviour under simulated intestinal digestion conditions (using methods described previously in Williams et al J. Pharm. Sci. (2012) 101, 3360-3380), the combination of the lipid based formulation and the ITZ IL is able to significantly enhance and maintain drug solubilisation in the aqueous solubilised phase when compared to an analogous formulation where ITZ FB was loaded at the same concentration, but in this case as a suspension since the lack of lipid solubility of the FB precluded formulation as a solution. Effective continued solubilisation of ITZ IL is consistent with the high absorption and systemic exposure seen in vivo.

1.-31. (canceled)

32. A lipid formulation of a poorly water soluble drug comprising a low melting ionic salt of the poorly water soluble drug, together with a substantially non-aqueous lipid vehicle.

33. The lipid formulation according to claim 32 wherein the low melting ionic salt is a ionic liquid salt of the poorly water soluble drug.

34. The lipid formulation according to claim 33 wherein the ionic liquid salt has a melting point of about 90° C. or less.

35. The lipid formulation according to claim 33 wherein the ionic liquid salt has a melting point of about 70° C. or less.

36. The lipid formulation according to claim 33 wherein the ionic liquid salt has a melting point of about 50° C. or less.

37. The lipid formulation according to claim 33 wherein the ionic liquid salt has a melting point of about 40° C. or less.

38. The lipid formulation according to claim 33 wherein the ionic liquid salt has a melting point of about 30° C. or less.

39. The lipid formulation according to claim 33 wherein the ionic liquid salt has a melting point of about 25° C. or less.

40. The lipid formulation according to claim 33 wherein the ionic liquid salt is an oil at room temperature.

41. The lipid formulation according to claim 32 wherein the low melting ionic salt of the poorly water soluble drug is at least twice as soluble in the non-aqueous lipid vehicle as the non-ionised drug.

42. The lipid formulation according to claim 41 wherein the low melting ionic salt of the poorly water soluble drug is at least 4-5 times as soluble in the non-aqueous lipid vehicle as the non-ionised drug.

43. The lipid formulation according to claim 32 wherein the poorly water soluble drug forms the cation of the low melting ionic salt and contains at least one basic ionisable nitrogen atom.

44. The lipid formulation according to claim 43 wherein the poorly water soluble drug forms a low melting ionic salt with an anion formed from carboxylic acids (RC(O)O⁻), phosphates (ROP(O)O₂⁻), phosphonates (RP(O)O₂⁻), sulfonates (RSO(O)₂O⁻), sulfates (ROS(O)₂O⁻), tetrazolys (R-tetrazolate) or bis(sulfonyl)imides (RSO₂—N⁻—SO₂R), where R is an optionally substituted hydrocarbon group having at least 2 carbon atoms.

45. The lipid formulation according to claim 44 wherein the poorly water soluble drug forms low melting ionic salt with an anion formed from phosphates (ROP(O)O₂⁻), or sulfates (ROS(O)₂O⁻).

46. The lipid formulation according to claim 43 wherein R is optionally substituted and is selected from the group consisting of an alkyl, alkenyl or alkynl group, each having from 4-40 carbon atoms, and a cycloalkyl or unsaturated cyclic hydrocarbon group, each having from 3-10 carbon atoms.

47. The lipid formulation according to claim 46 wherein R is an optionally substituted alkyl group having 4-24 carbon atoms.

48. The lipid formulation according to claim 32 wherein the poorly water soluble drug forms the anion of the low melting ionic salt and contains at least one acidic group.

49. The lipid formulation according to claim 48 wherein the poorly water soluble drug forms a low melting ionic salt with a cation selected from ⁺NR′₄ and ⁺PR′₄, wherein each R′ is independently selected from hydrogen and R″ where R″ is selected from the group of an alkyl, alkenyl or alkynl group each having from 4-40 carbon atoms and a cycloalkyl or unsaturated cyclic hydrocarbon group each having from 3-10 carbon atoms.

50. The lipid formulation of claim 32, wherein the substantially non-aqueous lipid vehicle comprises at least one oil or lipid.

51. The lipid formulation of claim 32, wherein the substantially non-aqueous lipid vehicle consists essentially of at least one oil or lipid.

52. The lipid formulation of claim 50, wherein the substantially non-aqueous lipid vehicle comprises at least one oil or lipid and at least one surfactant.

53. The lipid formulation of claim 51, wherein the substantially non-aqueous lipid vehicle comprises at least one oil or lipid, at least one surfactant and at least one co-solvent.

54. The lipid formulation of claim 32, wherein the substantially non-aqueous lipid vehicle comprises at least one surfactant and, optionally, at least one co-solvent.

55. The lipid formulation of claim 32, wherein the substantially non-aqueous lipid vehicle consists essentially of at least one surfactant and/or solvent, optionally with one or more co-surfactants or co-emulsifiers.

56. The lipid formulation of claim 32 consisting essentially of an ionic liquid salt of the poorly water soluble drug, together with one or more oils and/or liquids.

57. The lipid formulation of claim 32 consisting essentially of an ionic liquid salt of the poorly water soluble drug, together with one or more surfactants and/or solvents, optionally with one or more, co-surfactants or co-emulsifiers.

58. The lipid formulation of claim 32 in the form of a single phase.

59. Use of a lipid formulation according to claim 32 as a fill for a capsule.

60. A capsule, sachet, syringe or dropper device, ampoule, tube or bottle containing a lipid formulation according to claim 32.

61. A method for the manufacture of a lipid formulation of a poorly water soluble drug, according to claim 32, said method comprising the step of blending a low melting ionic salt of the poorly water soluble drug with a non-aqueous lipid vehicle.

62. The method of claim 61 wherein the resulting lipid formulation is a single phase liquid, solid or semi-solid.
